(19) United States
(12) Patent Application Publication
(10) Pub. No.: US 2004/0006104 A1
(43) Pub. Date: Jan. 8, 2004

(54) Neutrophil inhibitors to reduce inflammatory response

(75) Inventors: Rodney Dean Bush, Mason, OH (US); Paul Mitchell Hershberger, Maineville, OH (US); Judith Anne Young, Cincinnati, OH (US); Bhavani Kasibhatla, Hamilton, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH

(21) Appl. No.: 10/368,261

(22) Filed: Feb. 18, 2003

Related U.S. Application Data
(63) Continuation of application No. PCT/US01/25224, filed on Aug. 10, 2001.
(60) Provisional application No. 60/227,201, filed on Aug. 23, 2000.

(51) Int. Cl. A61K 31/4709; A61K 31/454; A61K 31/428; A61K 31/423; A61K 31/4184; A61K 31/405; C07D 417/02; C07D 413/02; C 07D 4 3/02

(52) U.S. Cl. 514/314; 514/323; 514/367; 514/375; 514/394; 514/414; 514/419; 546/176; 546/198; 546/199; 548/152; 548/217; 548/304.7; 548/304.4; 548/465; 548/509

(57) ABSTRACT

The invention provides novel compounds selected from the group consisting of:

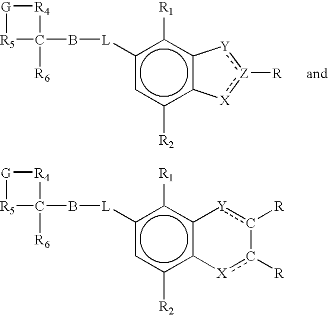

The compounds of the present invention are useful for the treatment and prevention of a variety of diseases and conditions associated with undesirable or abnormal inflammatory responses, such as ischemia-reperfusion injury. Accordingly, the invention further provides pharmaceutical compositions comprising these compounds. The invention still further provides methods of treatment or prevention for the above disorders using theses compounds or the compositions containing them.

### CROSS REFERENCE

This application is a continuation under 35 USC §120 of International Application PCT/US01/25224, with an international filing date of Aug. 10, 2001, and which claims the benefit of 35 USC §119(e)U.S. Provisional Application Serial No. 60/227,201, P&G Case No. 8223P, filed on Aug. 23, 2000.

### TECHNICAL FIELD

This invention relates to certain novel compounds which inhibit or reduce neutrophil activity by decreasing neutrophil migration to vascular endothelial cells. This invention further relates to compositions comprising these compounds as well as methods of using these novel compounds for the treatment of conditions which involve undesirable or abnormal inflammatory responses.

### BACKGROUND OF THE INVENTION

Neutrophils are an essential component of the host defense system against microbial invasion. In response to soluble inflammatory mediators released by cells at the site of injury, neutrophils emigrate into tissue from the bloodstream by crossing the blood vessel wall. At the site of injury, activated neutrophils kill foreign cells by phagocytosis and by the release of cytotoxic compounds, such as oxidants, proteases and cytokines. Neutrophils, however, can promote tissue damage themselves by releasing toxic substances at the vascular wall or releasing toxic substances into uninjured tissue. Also, neutrophils that stick to the capillary wall or clump in venules may produce tissue damage by ischemia. Such abnormal inflammatory responses have been implicated in the pathogenesis of a variety of clinical disorders.

Neutrophil adhesion at the site of inflammation occurs in two steps. Vascular endothelium adjacent to inflamed tissue upregulates adhesion molecules that may associate with neutrophils; neutrophils interact with the endothelium via low affinity adhesive mechanisms in a process known as “rolling”. The rolling of neutrophils along affected vascular endothelium is reported to be mediated by glycoproteins called selectins. In the second step, rolling neutrophils bind more tightly to vascular endothelial cells and migrate from the blood vessel into the tissue. This second step is mediated by integrins. Members of the leukocyte-specific CD18 family of integrins include Mac-1. Endothelial cell counter receptors for these integrins are the intercellular cell adhesion molecule, ICAM-1, a member of the immunoglobulin superfamily (Rothlein et al., 1986 J. Immunol. 137, 1270; Staunton et al., 1988 Cell 52, 925; Staunton et al., Nature 339, 61).

An important aspect of neutrophil mediated inflammation is the activation of endothelial cells and neutrophils. Staunton et al. reported that activation of endothelial cells results in an increase in the surface expression of ICAM-1 (Staunton et al., 1988 Cell 52, 925). The increased adhesion of neutrophils to vascular endothelium near tissue injury may be in part due to the increased expression of endothelial cell adhesion molecules.

Current clinical treatment approaches for the treatment of abnormal inflammatory response include steroids, catecholamines, prostaglandins, and NSAIDs (non-steroidal antiinflammatory drugs). Due to the generalized systemic effects of these agents, these agents often exhibit multiple side effects or other adverse drug reactions, as well as in some cases, inadequate efficacy.

Monoclonal antibodies have been reported to directly inhibit neutrophil adhesion to the vascular endothelium. These monoclonal antibodies recognize and block ligand-binding functions of some of these adhesive molecules including ICAM-1. Recombinant soluble adhesive molecules using Mac-1, are also reported in the literature. U.S. Pat. No. 5,708,141, issued Jan. 13, 1998, U.S. Pat. No. 5,747,296, issued May 5, 1998, U.S. Pat. No. 5,919,900, issued Jul. 6, 1999, and U.S. Pat. No. 5,789,178, issued Aug. 4, 1998, all to Corvas International, teach Neutrophil Inhibitory Factor (NIF), a glycoprotein isolated from nematodes (hookworm) or recombinant NIF, which inhibit neutrophil activity including adhesion to vascular endothelial cells. The usefulness of the above approaches, however, requires further evaluation. The above references are incorporated herein by reference in their entirety.

Despite the above technologies, a need still exists for potent, highly specific inhibitors of neutrophil activity, especially neutrophil migration to vascular endothelium as a treatment for undesirable or abnormal inflammatory response. Additional compounds which block adhesive molecules, are desirable for the treatment of inflammatory conditions. Small molecules, such as the compounds of the present invention, offer certain advantages over biological molecules. Smaller molecules generally have increased tissue penetration, lower immunogenicity, generally lower cost, and generally lower risks of serious adverse events.

### SUMMARY OF THE INVENTION

This invention relates to certain novel compounds which inhibit or reduce undesirable or abnormal inflammatory response by decreasing or inhibiting neutrophil activity, adhesion, and/or migration to vascular endothelial cells. This invention further relates to compositions comprising these compounds as well as methods of using these novel compounds for the treatment of conditions which involve undesirable or abnormal inflammatory responses. The compounds are selected from the group consisting of:

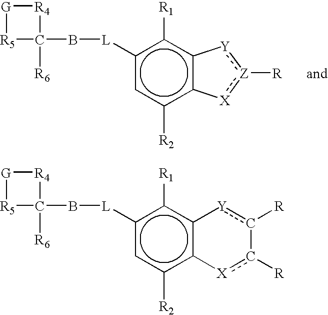

wherein R, R₁, R₂, R₄, R₅, R₆, B, L, G, X, Y, and Z are defined below.

This invention also includes optical isomers, diastereomers, and enantiomers of the formulas above, and mixtures thereof, and pharmaceutically-acceptable salts, hydrates, biohydrolyzable amides, esters, and imides thereof.

### DETAILED DESCRIPTION OF THE INVENTION

Terms and Definitions

“Alkyl” is a saturated or unsaturated hydrocarbon chain having 1 to 18 carbon atoms, preferably 1 to 12, more preferably 1 to 6, more preferably still 1 to 4 carbon atoms. Alkyl chains may be straight or branched. Preferred branched alkyl chains have one or two branches, preferably one branch. Preferred alkyl chains are saturated. Unsaturated alkyl chains have one or more double bonds and/or one or more triple bonds. Preferred unsaturated alkyl chains have one or two double bonds or one triple bond, more preferably one double bond. Alkyl chains may be unsubstituted or substituted with from 1 to 4 substituents. Preferred substituted alkyl chains are mono-, di-, or trisubstituted. The substituents may be lower alkyl chains, halo, hydroxy, aryloxy (e.g., phenoxy), acyloxy (e.g., acetoxy), carboxy, monocyclic or polycyclic aromatic ring (e.g., phenyl), monocyclic or polycyclic heteroaromatic ring, monocyclic or polycyclic carbocyclic aliphatic ring, monocyclic or polycyclic heterocyclic aliphatic ring, amide, and amino.

“Lower alkyl” is an alkyl chain comprised of 1 to 6, preferably 1 to 3 carbon atoms.

“Aromatic ring” is an aromatic hydrocarbon ring. Aromatic rings are monocyclic or fused bicyclic ring systems. Monocyclic aromatic rings contain from about 5 to about 10 carbon atoms, preferably from 5 to 7 carbon atoms, and most preferably from 5 to 6 carbon atoms in the ring. Bicyclic aromatic rings contain from 8 to 12 carbon atoms, preferably 9 or 10 carbon atoms in the ring system. Bicyclic aromatic rings include ring systems wherein one ring in the system is aromatic. Preferred bicyclic aromatic rings are ring systems wherein both rings in the system are aromatic. Aromatic rings may be unsubstituted or substituted with from 1 to 4 substituents on the ring. The substituents may be halo, cyano, alkyl, heteroalkyl, haloalkyl, phenyl, phenoxy, or any combination thereof. Preferred substituents include halo and haloalkyl. Preferred aromatic rings include naphthyl and phenyl. The most preferred aromatic ring is phenyl.

“Carbocyclic aliphatic ring” is a saturated or unsaturated hydrocarbon ring. Carbocyclic aliphatic rings are not aromatic. Carbocyclic aliphatic rings are monocyclic. Carbocyclic aliphatic rings contain from about 4 to about 10 carbon atoms, preferably from 4 to 7 carbon atoms, and most preferably from 5 to 6 carbon atoms in the ring. Carbocyclic aliphatic rings may be unsubstituted or substituted with from 1 to 4 substituents on the ring. The substituents may be halo, cyano, alkyl, heteroalkyl, haloalkyl, phenyl, phenoxy, or any combination thereof. Preferred substituents include halo and haloalkyl. Preferred carbocyclic aliphatic rings include cyclopentyl, cyclohexyl, cyclohexenyl, cycloheptyl, and cyclooctyl. More preferred carbocyclic aliphatic rings include cyclohexyl, cycloheptyl, and cyclooctyl.

“Halo” is fluoro, chloro, bromo, or iodo. Preferred halo are fluoro, chloro, and bromo; more preferred are chloro and fluoro, especially fluoro.

“Haloalkyl” is a straight, branched, or cyclic hydrocarbon substituted with one or more halo substituents. Preferred haloalkyl are C₁-C₁₂; more preferred are C₁-C₆; more preferred still are C₁-C₃. Preferred halo substituents are fluoro and chloro. The most preferred haloalkyl is trifluoromethyl.

“Heteroalkyl” is a saturated or unsaturated chain containing carbon and at least one heteroatom, wherein no two heteroatoms are adjacent. Heteroalkyl chains contain from 1 to 18 member atoms (carbon and heteroatoms) in the chain, preferably 1 to 12, more preferably 1 to 6, more preferably still 1 to 4. Heteroalkyl chains may be straight or branched. Preferred branched heteroalkyl chains have one or two branches, preferably one branch. Preferred heteroalkyl chains are saturated. Unsaturated heteroalkyl chains have one or more double bonds and/or one or more triple bonds. Preferred unsaturated heteroalkyl chains have one or two double bonds or one triple bond, more preferably one double bond. Heteroalkyl chains may be unsubstituted or substituted with from 1 to 4 substituents. Preferred substituted heteroalkyl chains are mono-, di-, or trisubstituted. The substituents may be lower alkyl, halo, hydroxy, aryloxy (e.g., phenoxy), acyloxy (e.g., acetoxy), carboxy, monocyclic aromatic ring (e.g., phenyl), monocyclic heteroaromatic ring, monocyclic carbocyclic aliphatic ring, monocyclic heterocyclic aliphatic ring, amide, and amino.

“Lower heteroalkyl” is a heteroalkyl chain comprised of 1 to 6, preferably 1 to 3, member atoms.

“Heteroaromatic ring” is an aromatic ring containing carbon and from 1 to about 4 heteroatoms in the ring. Heteroaromatic rings are monocyclic or fused bicyclic ring systems. Monocyclic heteroaromatic rings contain from about 5 to about 10 member atoms (carbon and heteroatoms), preferably from 5 to 7, and most preferably from 5 to 6 in the ring. Bicyclic heteroaromatic rings include ring systems wherein only one ring in the system is aromatic. Preferred bicyclic heteroaromatic rings are ring systems wherein both rings in the system are aromatic. Bicyclic heteroaromatic rings contain from 8 to 12 member atoms, preferably 9 or 10, in the ring. Heteroaromatic rings may be unsubstituted or substituted with from 1 to 4 substituents on the ring. The substituents may be hydroxy, amino, halo, cyano, alkyl, heteroalkyl, haloalkyl, phenyl, phenoxy, or any combination thereof. Preferred substituents include halo, haloalkyl, and phenyl. Preferred monocyclic heteroaromatic rings include thienyl, thiazolo, purinyl, pyrimidyl, pyridyl, and furanyl. More preferred monocyclic heteroaromatic rings include thienyl, furanyl, and pyridyl. The most preferred monocyclic heteroaromatic ring is thienyl. Preferred bicyclic heteroaromatic rings include benzo[β]thiazolyl, benzo[β]thiophenyl, quinolinyl, quinoxalinyl, benzo[β]furanyl, benzimidazolyl, benzoxazolyl, indolyl, and anthranilyl. More preferred bicyclic heteroaromatic rings include benzimidazolyl, benzo[β]thiazolyl, benzo[β]thiophenyl, and benzoxazolyl.

“Heteroatom” is a nitrogen, sulfur, or oxygen atom. Groups containing more than one heteroatom may contain different heteroatoms.

“Heterocyclic aliphatic ring” is a saturated or unsaturated ring containing carbon and from 1 to about 4 heteroatoms in the ring, wherein no two heteroatoms are adjacent in the ring and no carbon in the ring that has a heteroatom attached to it also has a hydroxyl, amino, or thiol group attached to it. Heterocyclic aliphatic rings are not aromatic. Heterocyclic aliphatic rings are monocyclic. Heterocyclic aliphatic rings contain from about 4 to about 10 member atoms (carbon and heteroatoms), preferably from 4 to 7 member atoms, and most preferably from 5 to 6 member atoms in the ring. Heterocyclic aliphatic rings may be unsubstituted or substituted with from 1 to 4 substituents on the ring. The substituents may be halo, cyano, alkyl, heteroalkyl, haloalkyl, phenyl, phenoxy or any combination thereof. Preferred substituents include halo and haloalkyl. Preferred heterocyclic aliphatic rings include piperzyl, morpholinyl, tetrahydrofuranyl, tetrahydropyranyl, and piperdyl.

“Phenyl” or “Ph” is a monocyclic aromatic ring which may or may not be substituted with from about 1 to about 4 substituents. The substituents may be fused but not bridged and may be substituted at the ortho, meta, or para position on the phenyl ring, or any combination thereof. The substituents may be halo, acyl, cyano, alkyl, heteroalkyl, haloalkyl, phenyl, phenoxy, or any combination thereof. Preferred substituents on the phenyl ring include halo and haloalkyl. The most preferred substituent is halo. The preferred substitution pattern on the phenyl ring is ortho or meta. The most preferred substitution pattern on the phenyl ring is meta.

Compounds

The invention involves compounds having the following structure:

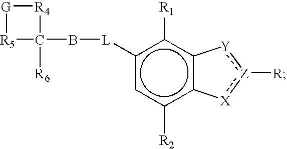

wherein X and Y are heteroatoms wherein at least X or Y is nitrogen wherein the nitrogen can be unsubstituted or substituted with a lower alkyl group; in another embodiment both X and Y are nitrogen; in yet another embodiment both X and Y are nitrogen and one nitrogen is substituted with a C₁-C₄ alkyl group;

Z is a carbon atom, two carbon atoms or a heteroatom; if Z is two carbon atoms, the structure is:

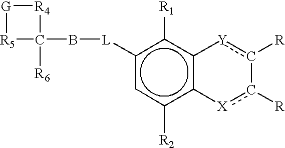

in another embodiment Z is a single carbon atom;

R is independently selected from the group consisting of alkyl, lower alkyl, aromatic ring, carbocyclic aliphatic ring, halo, haloalkyl, heteroalkyl, lower heteroalkyl, heteroaromatic ring, heterocyclic aliphatic ring, hydrogen, —OH, —NH₂, —SH, or —OCH₃; in another embodiment R is selected from the group consisting of alkyl, lower alkyl, aromatic ring, carbocyclic aliphatic ring, halo, haloalkyl, heteroalkyl, lower heteroalkyl, heterocyclic aliphatic ring, heteroaromatic ring, hydrogen, —OH, —NH₂, or —OCH₃; in another embodiment R is selected from the group consisting of alkyl, lower alkyl, aromatic ring, carbocyclic aliphatic ring, halo, haloalkyl, heteroalkyl, lower heteroalkyl, hydrogen, —OH, —NH₂, —OCH₃; in yet another embodiment R is alkyl, lower alkyl, halo, haloalkyl, hydrogen, —OH, —OCH₃, —NH₂; in yet another embodiment R is hydrogen, NH₂, or a C₁-C₄ alkyl group;

R₁ and R₂ are, independently, selected from the group consisting of alkyl, lower alkyl, aromatic ring, carbocyclic aliphatic ring, halo, haloalkyl, heteroalkyl, lower heteroalkyl, heteroaromatic ring, heterocyclic aliphatic ring, or hydrogen; in another embodiment R₁ and R₂ are, independently, selected from the group consisting of lower alkyl, hydrogen, heteroalkyl, a 5 or 6-membered heterocyclic aliphatic ring, heteroalkyl, a branched or nonbranched alkyl heteroaromatic ring, or phenyl group; in another embodiment R₁ and R₂ are, independently, selected from the group consisting of lower alkyl, hydrogen, heteroalkyl, a branched alkyl heteroaromatic ring wherein the alkyl chain contains a heteroatom, or a phenyl group;

L is selected from the group consisting of

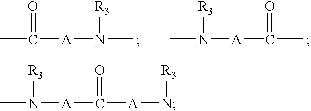

in another embodiment L is

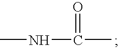

wherein A is selected from the group consisting of a branched or unbranched alkyl, a branched or unbranched lower alkyl, or A is a covalent bond; in another embodiment A is a C₁-C₄ alkyl; in a another embodiment A is an unbranched C₁-C₂ alkyl;

R₃ is selected from the group consisting of alkyl, lower alkyl, aromatic ring, carbocyclic aliphatic ring, haloalkyl, heteroalkyl, lower heteroalkyl, heterocyclic aliphatic ring, or hydrogen; in another embodiment R₃ is lower alkyl or hydrogen; in even another embodiment R₃ is hydrogen;

B is selected from the group consisting of alkyl, lower alkyl, haloalkyl, heteroalkyl, lower heteroalkyl or B is a covalent bond; in another embodiment B is lower alkyl, lower heteroalkyl, or a covalent bond; in yet another embodiment B is a C₁-C₄ alkyl, C₁-C₄ heteroalkyl interrupted with an oxygen atom, or a covalent bond;

G is nil, or a substituent that links R₄ and R₅ into a cyclic ring structure which may be a 5-10 atom aromatic, aliphatic, heteroaromatic, and/or heteroaliphatic ring structure, which is unsubtituted or substituted wherein R₄ is a substituent at any position on the ring structure; in another embodiment G is nil;

if G is nil then, R₄ and R₅ are as follows:

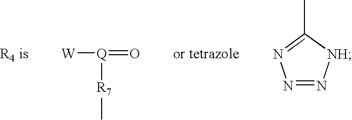

wherein Q is selected from the group consisting of a carbon atom,

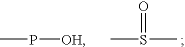

in another embodiment Q is a carbon atom;

W is selected from the group consisting of —OH, —NHOH; in another embodiment W is an —OH group;

R₇ is selected from the group consisting of alkyl, lower alkyl, aromatic ring, heteroaromatic ring, carbocyclic aliphatic ring, heteroalkyl, lower heteroalkyl, heterocyclic aliphatic ring, hydrogen or a covalent bond; in another embodiment R₇ is phenyl, C₁-C₄ alkyl, hydrogen, or a covalent bond; in yet another embodiment R₇ is methyl, hydrogen or a covalent bond;

R₅ is selected from the group consisting of hydrogen, alkyl, lower alkyl, aromatic ring, carbocyclic aliphatic ring, halo, haloalkyl, heteroalkyl, lower heteroalkyl, heterocyclic aliphatic ring, —C₂H₄— aromatic ring, —C₂H₄-carbocyclic aliphatic ring, —C₂H₄-heterocylic aliphatic ring, —C₂H₄—Ph, —CH₂-aromatic ring, —CH₂-carbocyclic aliphatic ring, —CH₂-heterocyclic aliphatic ring, —CH₂Ph; in another embodiment R₅ is a carbon atom, —C₂H₄— aromatic ring, —C₂H₄-carbocyclic aliphatic ring, —C₂H₄-heterocyclic aliphatic ring, —C₂H₄—Ph, —CH₂-aromatic ring, —CH₂-carbocyclic aliphatic ring, —CH₂-heterocyclic aliphatic ring, —CH₂Ph; or a phenyl group; in yet another embodiment R₅ is —C₂H₄—Ph or —CH₂Ph wherein the phenyl group is substituted with an —OAc group or a phenyl group;

R₆ is selected from the group consisting of alkyl, lower alkyl, aromatic ring, carbocyclic aliphatic ring, halo, haloalkyl, heteroalkyl, lower heteroalkyl, heterocyclic aliphatic ring, hydrogen, or any R₄ group; in another embodiment R₆ is hydrogen;

in another embodiment if R is —SH then R₅ is selected from the group consisting of C₁-C₁₇ alkyl, hydrogen, lower alkyl, aromatic ring, carbocyclic aliphatic ring, halo, haloalkyl, heteroalkyl, lower heteroalkyl, heterocyclic aliphatic ring, —C₂H₄— aromatic ring, —C₂H₄-carbocyclic aliphatic ring, —C₂H₄-heterocyclic aliphatic ring, —C₂H₄—Ph, —CH₂-aromatic ring, —CH₂-carbocyclic aliphatic ring, —CH₂-heterocyclic aliphatic ring, —CH₂Ph and R₆ is selected from the group consisting of C₁-C₁₇ alkyl, lower alkyl, aromatic ring, carbocyclic aliphatic ring, halo, haloalkyl, heteroalkyl, lower heteroalkyl, heterocyclic aliphatic ring, hydrogen, or any R₄ group;

optical isomers, diastereomers, and enantiomers of the formula above, and mixtures thereof, and pharmaceutically-acceptable salts, hydrates, biohydrolyzable amides, esters, and imides thereof.

Synthesis of Compounds

Compounds useful in the subject invention can be made using conventional organic synthesis. The starting materials used in preparing the compounds of the invention are known, made by known methods, or are commercially available. A particularly preferred synthesis is the general reaction sequence (Scheme 1) shown below:

An amino acid is esterified or linked to a solid support to give 1. The protecting group is chosen such that it is compatible with the overall chemistry route and can be removed late in the synthesis. This compound is reacted with a carboxylic acid 2, which is previously protected if necessary, under amide bond forming conditions to give 3. The amide is typically formed after activation of the carboxylic acid using a standard reagent system such as a carbodiimide. The intermediate 3 is subsequently deprotected, and cleaved from resin in solid phase cases, to give the title compounds indicated by 4. The specific reaction conditions for several examples are disclosed below.

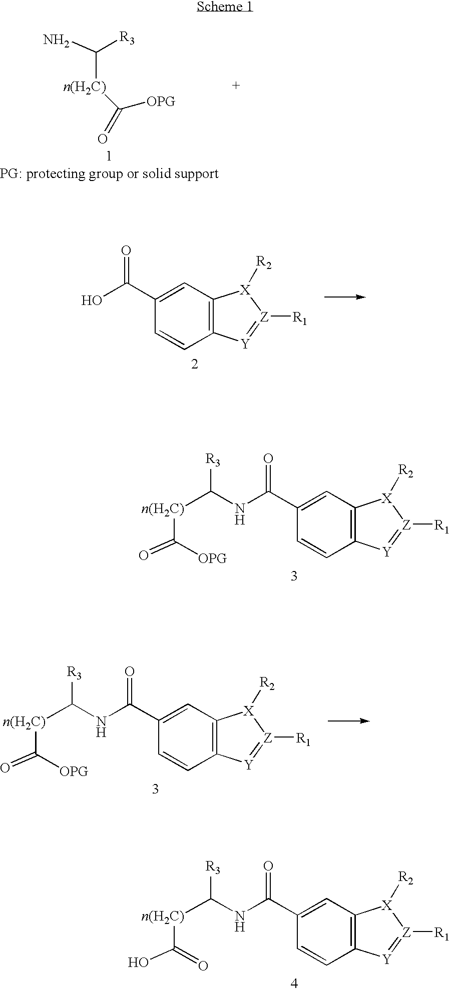

The R groups used to illustrate the reaction schemes do not correlate to the R groups used to describe the various moieties of the formula for the compounds. For example, R₁ in the formulas does not represent the same moiety as R₁ in this section. The following non-limiting examples illustrate the compounds, compositions, and uses of the invention.

### EXAMPLES

Compounds are analyzed using ¹H and ¹³C NMR analysis, mass spectroscopy, high performance liquid chromatography (HPLC), and elemental analysis. Typically, inert solvents are used, preferably in dry form. Normal phase chromatography is performed on silica gel (70-230 mesh, Aldrich, or 230-400 mesh, Merck) as appropriate. Reverse phase chromatography is performed using standard HPLC conditions.

The following general procedure is provided for a solid phase variation of the key intermediate 1. In this procedure, an Fmoc protected amino acid of choice is loaded onto chlorotrityl resin, and then the Fmoc protecting group is removed. The resulting resin bound materials are used in coupling reactions with intermediates such as 2a-2j.

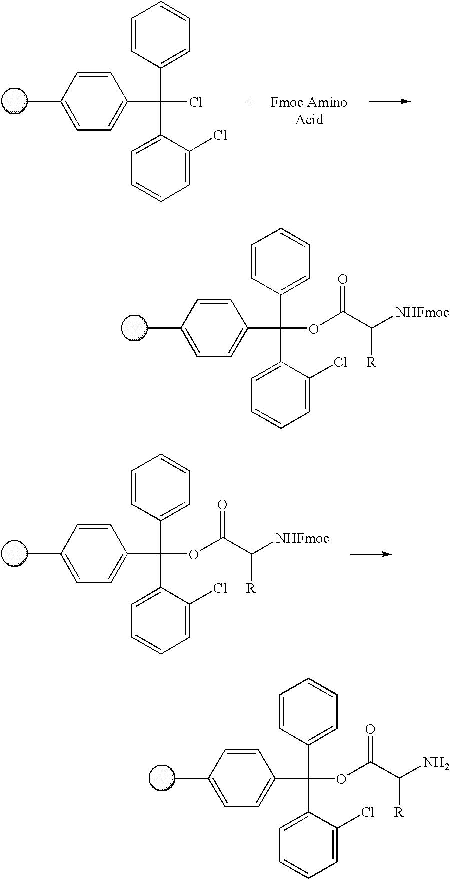

The 2-chlorotrityl chloride resin is preswollen in anhydrous dichloromethane (DCM). The Fmoc amino acid (1.5 eq) is dissolved in DCM. If the acid does not dissolve completely a small amount of N,N-dimethylformamide (DMF) is added. The resin is treated with the acid solution and diisopropylethylamide (DIPEA) (1.5 eq). The mixture is agitated with a shaker for 24 hours. The resin is filtered and washed three times with DCM, twice with DMF, twice with DCM, and three times with methanol. It is then dried in vacuo.

The resin is preswollen in anhydrous DCM and treated twice with a 20% solution of piperidine in DMF, and then left to agitate for two 2 hour periods. The resin is filtered and washed three times with DCM, twice with DMF, twice with DCM, and three times with methanol. It is then dried in vacuo. To ensure complete removal of the Fmoc protecting group, the resin is analyzed using infrared spectroscopy.

The following procedures describe the preparation of the key intermediates 2a-2j.

2a: 5-Benzimidazolecarboxylic acid, Commercially Available

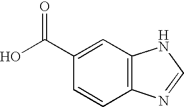

2b: 6-Quinoxalinecarboxylic acid, Commercially Available

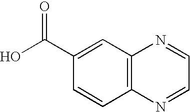

2c: Benzimidazol-1,5-dicarboxylic acid 1-tert-butyl ester

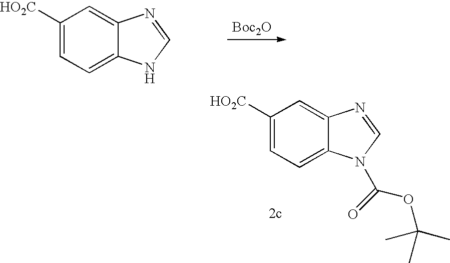

Di-tert-butyl dicarbonate (4.72 g, 1.1 equivalents) is added to benzimidazole-5-carboxylic acid (3.19 g, 1 equivalent) in DMF (100 ml). After 24 hours, the solvent is removed in vacuo and the residue is purified by flash chromatography, eluting with ethyl acetate. Although separable, the regioisomers are isolated together to afford 2c (2.31 g).

2d: 2-tert-butoxycarbonylamino-1H-benzimidazole-5-carboxylic acid

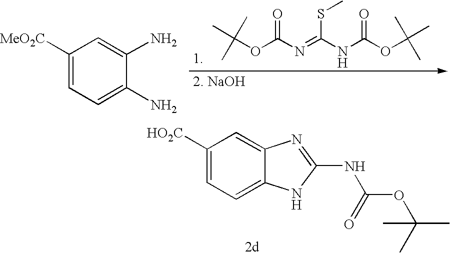

Methyl 3,4-diaminobenzoate (7.0 g, 1 equivalent), 1,3-bis(tert-butoxycarbonyl)-2-methyl-2-thiopseudourea (19.6 g, 1.6 equivalents) and camphorsulfonic acid (200 mg, cat.) are heated at reflux in ethanol (200 ml) for 3 h. The mixture is allowed to cool to r.t., then the solid is filtered, washed with ethanol, and dried to afford the intermediate methyl ester (8.2 g).

Sodium hydroxide (5.6 g, 5 equivalents) is added cautiously to 2-tert-butoxycarbonylamino-1H-benzimidazole-5-carboxylic acid methyl ester (8.2 g, 1 equivalent) in a mixture of methanol (80 ml) and water (70 ml). The reaction is heated to 40° C. for 4 h, then allowed to cool to r.t., acidified with conc HCl (20 ml, 7 equivalents), and cooled in ice. The solid is filtered, washed with the minimum amount of water (2×20 ml), and dried to afford the 2d (7.63 g).

2e: 2-Methyl-benzimidazole-1,5-dicarboxylic acid 1-tert-butyl ester

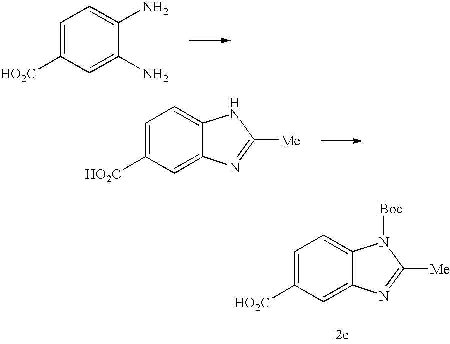

3,4-Diamino-benzoic acid (10 g), amberlyst (200 mg), triethylorthoacetate (29.5 mL) and ethanol (100 mL) are added to the reaction vessel. The mixture is stirred at room temperature overnight and the progress monitored by LC/MS. The mixture is concentrated and treated with 2M HCl (0.5 mL) in tetrahydrofuran (THF) (20 mL), and is then stirred at room temperature for 15 minutes. The reaction is filtered and concentrated to afford the intermediate acid as a solid (8.44 g).

2-methyl-1H-benzimidazole-5-carboxylic acid (8.44 g) is treated with di-tert-butyl dicarbonate (11.5 g) and stirred at room temperature in DMF (200 mL) for 24 hours. The mixture is concentrated, the residue taken up in ethyl acetate and washed with water (10 mL). The organic phase is dried (MgSO₄) and concentrated to afford the 2e as a solid (5.65 g).

2f: 3-methyl-3H-benzimidazole-5-carboxylic acid

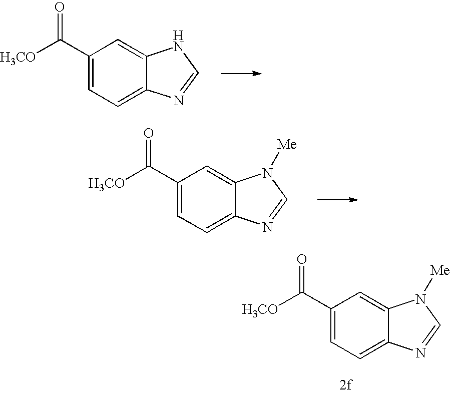

Sodium hydride (0.55 g) is washed free of oil by washing with anhydrous petroleum ether under a stream of nitrogen. It is suspended in dimethylformamide (10 mL), and 3H-benzimidazole-5-carboxylic acid methyl ester is added as a solution in the remaining dimethylformamide (90 mL). The solution is stirred for 30 minutes. Iodomethane (1.6 g) is added and the reaction monitored by LC/MS. The dimethylformamide is evaporated in vacuo and the residue gradually poured onto water. The solution is taken up in ethyl acetate and washed with brine. The organic phase is dried (MgSO₄), concentrated, and then chromatographed on silica (5% MeOH/DCM) to afford the N-methyl intermediate as a white solid (1.75 g).

3-methyl-3H-benzimidazole-5-carboxylic acid methyl ester (1.75 g) is dissolved in 1:1 MeOH/water (16 mL) and treated with lithium hydroxide added (386.3 mg). The reaction is stirred at room temperature for 40 hours and progress monitored by LC/MS. The solution is acidified with 2M HCl to pH 7. The residue is taken up in ethanol, filtered, and concentrated in vacuo. This afforded 2f as a solid (1.0 g).

2g: 2-methoxy-1H-benzimidazole-5-carboxylic acid

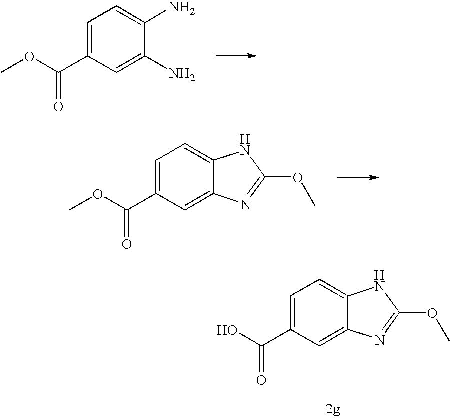

Methyl 3,4-diaminobenzoate (1.66 g, 10 mmol) is suspended in tetramethyl orthocarbonate (2.72 g, 20 mmol), and acetic acid (AcOH) (5 ml) is added and the reaction is heated at 100° C. for 4 hrs. The reaction is diluted with ethylacetate (EtOAc) (100 ml) and saturated NaHCO₃ (100 ml). The insoluble precipitate is removed by filtration and the EtOAc is dried over MgSO₄, filtered, and concentrated. The product is purified by column chromatography, eluting with 70/30 EtOAc/hexane and then with 100% EtOAc to yield the intermediate (380 mg).

The methyl ester (380 mg, 1.84 mmol) is dissolved in MeOH (50 ml) and water (20 ml), and treated with LiOH.H₂O (400 mg, 9.53 mmol). The reaction is heated to 50° C. for 5 days and then cooled and concentrated in vacuo. The residue is diluted with water (30 ml) and washed with EtOAc (30 ml). The aqueous phase is taken to pH 2 with 1M HCl and extracted with EtOAc (3×20 ml). The combined organic phases are dried over MgSO₄, filtered, and concentrated to yield 2g (283 mg).

2h: Benzothiazole-6-carboxylic acid, Commercially Available

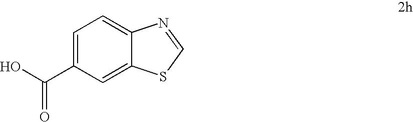

2i: 2-hydroxybenzoxazole-5-carboxylic acid.

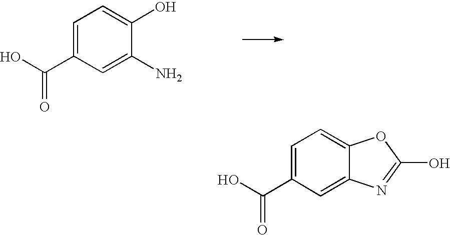

3-Amino-4-hydroxybenzoic acid (3 g, 19.59 mmol) is dissolved in THF (200 ml) and 1,1-carbonyldiimidazole (6.66 g, 41.11 mmol) is added in a single portion. The reaction is stirred for 1 hr. Water (200 ml) is added and, after 10 minutes, the reaction is acidified to pH 2 with 2M HCl. The reaction is then extracted with Et₂O (3×200 ml) and the combined organic phases are dried over MgSO₄, filtered, and concentrated in vacuo to yield 2i (3.48 g).

2j: 2-Amino-benzoxazole-5-carboxylic acid.

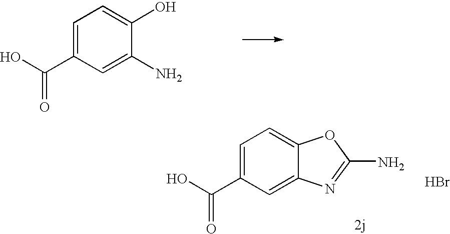

The amino alcohol (735 mg, 4.8 mmol) is dissolved in EtOH (20 ml) and cyanogen bromide (1.91 ml of 5M soln in MeCN) is added dropwise. The reaction is stirred overnight and more cyanogen bromide (1.91 ml of 5M soln in MeCN) is added. After stirring for a further 72 hrs the reaction is concentrated and the residue is triturated with Et₂O and the solid is dried under vacuum to yield 1.16 g of the product.

The following are examples of specific compounds prepared from intermediates 2a-2j.

### Examples Prepared from 2a

### Example I and Example II

### 2-(benzimidazol-6-ylcarbonylamino)-3-phenylpropanoic acid and sodium 2-(benzimidazol-6-ylcarbonylamino)-3-phenylpropionate

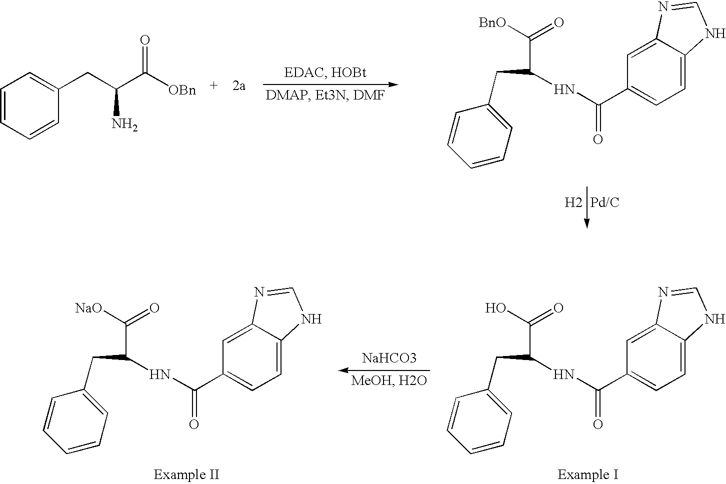

L-phenylalanine benzyl ester p-toluene sulfonate salt (10.28 g, 24.05 mmole, MW=427.5) is dissolved in 60 ml anhydrous DMF. Et₃N (3.21 ml, 2.33 g, d=0.726, 24.18 mmole) is added to the solution. The mixture is stirred at r.t. with positive N₂ for 10 min., followed by addition of 2a (3 g, 18.5 mmole, MW=162.15), 1-ethyl-3-(3-dimethyl amino-propyl) carbodiimide HCl salt (EDAC, 5.32 g, 27.75 mmole, MW=191.7), 1-hydroxybenzotriazole HCl salt (HOBt, 3.75 g, 27.75 mmole, MW=135.13), dimethylaminopyridine (DMAP, 0.24 g, 2 mmole, MW=122.17), and Et₃N (3.21 ml, 2.33 g, d=0.726, 24.18 mmole). The mixture is stirred at r.t. for 20 hrs under positive N₂. The reaction is then poured into a beaker which contains 300 ml H₂O and 300 ml EtOAc. The EtOAc layer is collected and washed with saturated NaHCO₃ (3×250 ml), 0.4N HCl (3×250 ml), and saturated NaCl (2×200 ml). The EtOAc layer is then dried with anhydrous Na₂SO₄, filtered, and evaporated to give 5.7 g of the crude product. Purification is achieved by chromatography, eluting with 3% methanol in methylene chloride. This returns 4.5 g of phenylmethyl (2S)-2-(benzimidazol-5-ylcarbonylamino)-3-phenylpropanoate with R_{f}=0.25 (3% MeOH:CH₂Cl₂).

Phenylmethyl (2S)-2-(benzimidazol-5-ylcarbonylamino)-3-phenylpropanoate (500 mg, 1.25 mmole, MW=399) is mixed with 100 mg Pd/C and 200 ml MeOH. The solution is stirred at r.t. under an H₂ filled balloon for 14 hrs. The reaction mixture is filtered through Celite and evaporated to give an oily residue. MeOH (2 ml) is added to the oil, followed by 50 ml of ethyl ether. The resulting precipitate is collected to give 300 mg of Example I as a solid.

Phenylmethyl (2S)-2-(benzimidazol-5-ylcarbonylamino)-3-phenylpropanoate (500 mg, 1.25 mmole, MW=399) is mixed with 100 mg Pd/C and 200 ml MeOH. The solution is stirred at r.t. under an H₂ filled balloon for 14 hrs. The reaction mixture is filtered through Celite and evaporated to give an oily residue. MeOH (2 ml) is added to the oil, followed by 1 eq. of NaHCO₃ in 5 ml H₂O. The solution mixture is evaporated to give Example II as a solid.

### Example III

### (2S)-2-(Benzimidazol-5-ylcarbonylamino)-3-(O-acetyl-p-hydroxyphenyl)propanoic acid

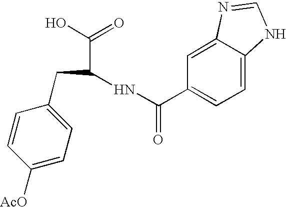

O-Acetyl-L-tyrosine benzyl ester trifluoroacetic acid salt: O-Acetyl-N-BOC-L-tyrosine benzyl ester (247.5 mg, 0.60 mmol) is dissolved in dry dichloromethane (5 mL) under argon. Triethylsilane (0.19 mL, 1.20 mmol) is added and the reaction mixture is cooled to 0° C. Trifluoroacetic acid (0.46 mL, 6.0 mmol) is then added via syringe and the resulting solution is stirred at 0° C. for 1 hour. The reaction mixture is concentrated via rotary evaporation to a thick oil and then further dried under vacuum overnight. A quantitative yield is assumed and this compound is used as is in the coupling reaction that follows.

Benzyl (2S)-2-(benzimidazol-5-ylcarbonylamino)-3-(O-acetyl-p-hydroxyphenyl)propanoate: A flask containing O-acetyl-L-tyrosine benzyl ester trifluoroacetic acid salt (255.8 mg, 0.60 mmol) and equipped with a stir bar and septum is flushed with argon. The oil is dissolved in anhydrous DMF (3 mL) and triethylamine (92 μL, 0.66 mmol). After stirring this solution for about 10 minutes, 2a (97.9 mg, 0.60 mmol), 1-(3-dimethylaminopropyl)-3-ethylcarbodiimide hydrochloride (126.5 mg, 0.66 mmol), 1-hydroxybenzotriazole hydrate (89.2 mg, 0.66 mmol), 4-dimethylaminopyridine (8 mg, 0.06 mmol), and triethylamine (92 μL, 0.66 mmol) are added and the resulting slurry is stirred at room temperature overnight. The reaction mixture is diluted with water (30 mL) and ethyl acetate (30 mL) and the layers are separated. The organic layer is washed with saturated sodium bicarbonate solution (3×25 mL) and brine (2×25 mL) and dried over MgSO₄. Concentration via rotary evaporation gives a yellow solid which is applied to a FlashElute 40S column of silica gel in 3% methanol/dichloromethane. The eluent polarity is gradually increased to 5% methanol/dichloromethane to elute the desired product cleanly. After concentration of the appropriate fractions, 62.4 mg (23% over two steps) of the desired product is obtained.

(2S)-2-(Benzimidazol-5-ylcarbonylamino)-3-(O-acetyl-p-hydroxyphenyl)propanoic acid: Benzyl (2S)-2-(benzimidazol-5-ylcarbonylamino)-3-(O-acetyl-p-hydroxyphenyl)propanoate (62.4 mg, 0.14 mmol) and 30% Pd/C (13.8 mg) are placed in a small flask equipped with a stir bar and septum and flushed with argon. Anhydrous methanol (2.5 mL) is added via syringe and the argon line is replaced with a hydrogen balloon. The resulting mixture is stirred under hydrogen overnight at room temperature. The reaction mixture is filtered through a pad of Celite®, which is washed with methanol. The filtrate is concentrated via rotary evaporation to a solid which is re-dissolved in a minimum of methanol and triturated with ether to give 30.8 mg of the Example III as a solid.

### Examples Prepared from 2b

### Example IV

### (2S)-3-Phenyl-2-[(1-quinoxalin-6-ylmethanoyl)amino]propionic acid

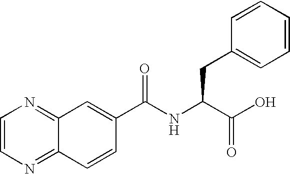

(2S)-3-Phenyl-2-[(1-quinoxalin-6-ylmethanoyl)amino]propionic acid, t-butyl ester: Quinoxaline-6-carboxylic acid (2b, 2.57 g, 14.8 mmol) is dissolved in anhydrous dichloromethane (75 mL) and N,N-diethyl-isopropylamine (2.83 mL, 16.2 mmol) under argon in a flask equipped with stir bar and septum. 1-(3-dimethylaminopropyl)-3-ethylcarbodiimide hydrochloride (3.11 g, 16.2 mmol) is added and allowed to stir 10 minutes before 1-hydroxybenzotriazole hydrate (2.19 g, 16.2 mmol) is added. After another 5 minutes, L-phenylalanine t-butyl ester hydrochloride (3.80 g, 14.8 mmol) is added and the mixture is allowed to stir at room temperature overnight. The reaction mixture is diluted with dichloromethane (100 mL) and washed with saturated sodium bicarbonate solution (1×100 mL), water (1×100 mL), and brine (1×100 mL). The organic layer is dried over MgSO₄ and concentrated to a brown foam which is applied to a column of silica gel in dichloromethane and eluted with 2% methanol/dichloromethane. Concentration of the appropriate fractions gives 4.4 g (79%) of the desired product as a very thick oil.

(2S)-3-Phenyl-2-[(1-quinoxalin-6-ylmethanoyl)amino]propionic acid: (2S)-3-Phenyl-2-[(1-quinoxalin-6-ylmethanoyl)amino]propionic acid, t-butyl ester (4.4 g, 11.7 mmol) is dissolved in 4M HCl in dioxane under argon (whereupon it rapidly turns green) and allowed to stir at room temperature overnight. The resulting slurry is concentrated to a solid and dissolved in saturated sodium bicarbonate solution. This is washed once with ethyl acetate (ethyl acetate layer is discarded) and re-acidified with 1N HCl to give a slurry which is extracted with ethyl acetate until no longer cloudy. The combined ethyl acetate layers are dried over MgSO₄ and concentrated to give a brown solid. The solid is redissolved in saturated sodium bicarbonate solution and filtered. The filtrate is concentrated to a solid and then slurried in methanol and refiltered. The filtrate is concentrated to a minimum of methanol and triturated with ether to give a solid after filtration. The solid is dissolved in water (200 mL) and acidified with 1N HCl (10 mL) to give a thick slurry which is filtered and washed with water to give 1.88 g of the Example IV as a solid after drying under vacuum.

### Examples Prepared from 2c

### Example V

### 2-(benzimidazol-6-ylcarbonylamino)-2-phenylacetic acid

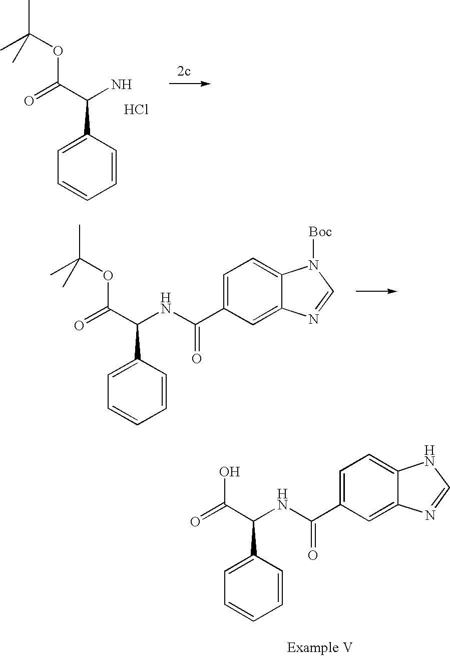

To 90 ml of methylene chloride are added 2.29 g of 2c. This dissolves to give an orange solution after 2.43 ml of triethylamine are added. While stirring, 2.2 g of the protected amino acid are added. This is followed by the addition of 3.03 g of HOBt and 3.52 g of EDC in that order. The solution is stirred over four nights. The solution is then washed with 10% citric acid, water, sodium bicarbonate, and brine. The organic layer is then dried over sodium sulfate, and the solvent removed to afford 3.84 g of the doubly protected intermediate as an orange oil.

The intermediate (3.84 g) is dissolved in 22 ml of dioxane and treated with 38 ml of 4M HCl in dioxane. This is allowed to stir overnight, during which the product precipitates. The solid is filtered, washed with ether, dried in a vacuum oven, and then is recrystallized from methanol/ether to give 500 mg of pure product and additional crude material containing methyl ester. The crude isolate can be acidified and extracted with ethyl acetate. Concentration of the aqueous part until solid formation begins affords an additional crop of pure material.

### Example VI

### (S)-[(1H-benzimidazol-5-yl-methanoyl)-amino]-2-phenyl-acetic acid

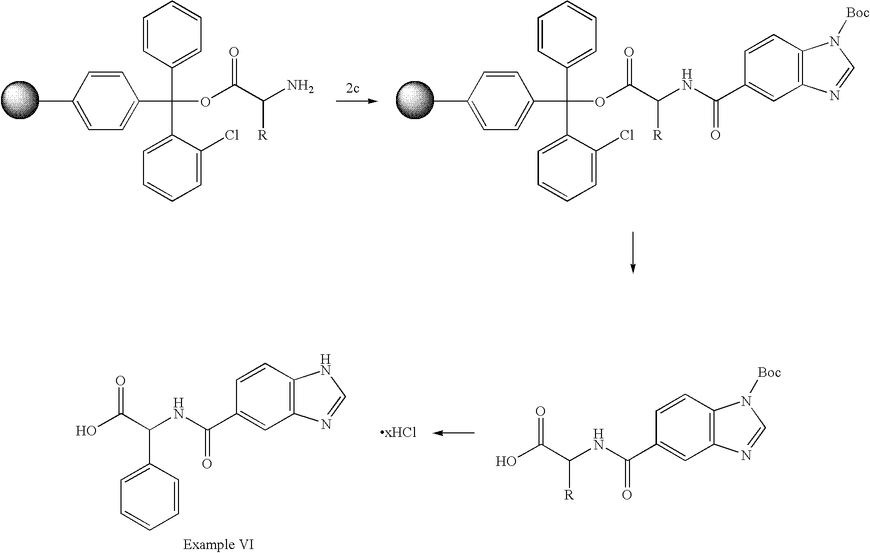

Compound 2c (1.8 g) is dissolved in N-methylpyrrolidinone (NMP) (19 ml). Aliquots of this mixture (1 ml, 3 equivalents) are then added to the amino acid resin (100 mg, 1 equivalent) followed by diisopropylethylamine (125 ml, 6 equivalents). PyBroP (3.2 g) is dissolved in NMP (19 ml) and aliquots (1 ml, 3 equivalents) are added to the amino acid resin. The mixture is shaken for 48 h, and then filtered and the resin washed with DMF, DCM and MeOH. This washing sequence is repeated two times, then the resin is washed with DCM, MeOH and diethyl ether, this sequence being repeated twice. The degree of coupling is qualitatively assessed by Kaiser tests, and in most cases a single coupling is sufficient.

The resin is treated with a mixture of AcOH, trifluoroethanol and DCM (1:1:8, 2 ml) for 1 h. After filtration, the resin is rinsed with DCM and subjected to a second cleavage for 1 h. The filtrates are combined and concentrated in vacuo. Hexane is added and the mixture is concentrated again to azeotropically remove residual acetic acid. This affords 40 mg of the cleaved intermediate.

The BOC protected regioisomers are treated with 4M HCl in dioxane (1 ml) for 24 h. Concentration and trituration of the residue with DCM gives 28.4 mg of Example VI.

### Example VII

### (S)-2-[(1-1H-Benzimidazol-5-yl-methanoyl)-amino]-4-phenyl-butyric acid hydrochloride

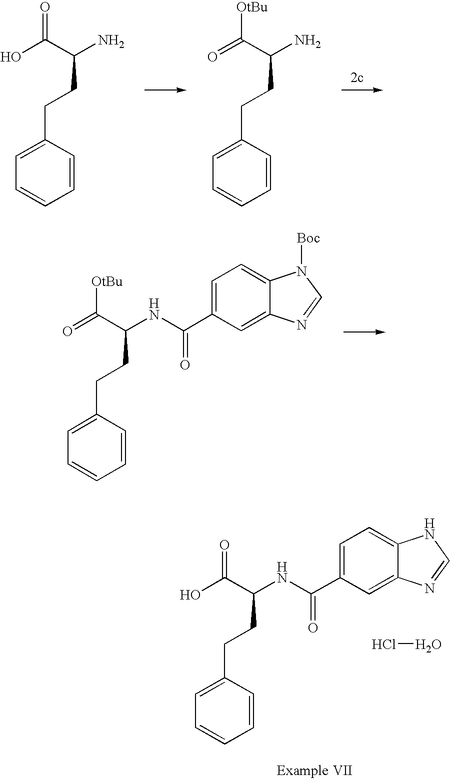

A solution of the amino acid (5.0 g) in dioxane (25 ml) is treated with sulfuric acid (3.1 ml) and stirred. Another flask is then fitted with a dry-ice condenser to enable the collection of isobutylene reagent at −78° C. The solution of starting material is then cooled to −78° C. and the cold liquid isobutylene is added. The mixture is stirred at room temp in a pressure bottle for 3 days. The bottle is then cooled to −78° C. and opened. It is then stirred while open and allowed to warm up in an ice bath. After 20 minutes of stirring, 55 ml of 2N NaOH are added slowly at 0° C. The product is extracted with ether and washed with dilute sodium bicarbonate. The solution is dried over sodium sulfate and the solvent is removed to give 4.9 g of the t-butyl ester as a yellow oil.

To 100 ml of methylene chloride are added 2.50 g of 2c. This dissolves to give a clear orange solution after 2.66 ml of triethylamine are added. While stirring, 2.33 g of the amino t-butyl ester are added. This is followed by the addition of 3.33 g of HOBt and 3.86 g of EDC in that order. The solution is stirred over two nights. The solution is then washed with 10% citric acid, water, sodium bicarbonate, and brine. The organic layer is dried over sodium sulfate and the solvent removed to afford 3.90 g of the coupled intermediate as an orange foam.

The coupled intermediate (2.78 g) is dissolved in 15 ml of dioxane and treated with 27 ml of 4M HCl in dioxane. After stirring overnight, the product comes out of solution. The solid is filtered, washed with ether, and dried in a vacuum oven. The solid is then taken up in water and some impurity is extracted with ethyl acetate. The aqueous solution is lyophilized to give 1.4 g of a tan solid. This is recrystallized twice from acetonitrile and water to give 750 mg of Example VII as solid.

Example VIII

### (S)-2-[(1H-benzimidazol-5-yl-methanoyl)-amino]-4-phenylbutyric acid

The procedures used to prepare Example VI are used to prepare 28.4 mg of Example VIII, substituting the appropriate resin bound amino acid starting material.

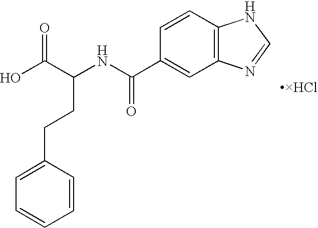

### Example IX

### (S)-2-[(1-1H-Benzimidazol-5-yl-methanoyl)-amino]-3-thiophen-2-yl-propionic acid

The procedures used to prepare Example VI are used to prepare 39.6 mg of Example IX, substituting the appropriate amino acid starting material.

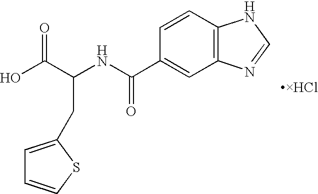

### Example X

### (S)-2-[(1-1H-Benzimidazol-5-yl-methanoyl)-amino]-3-cyclohexyl-propionic acid

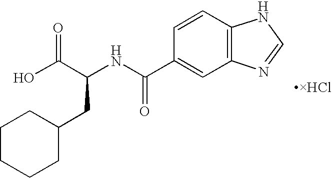

Example X is prepared using the procedures described for the preparation of Example VI, substituting the appropriate amino acid starting material. This provides 41.4 mg of the product.

### Example XI

### 3-[(1-1H-Benzimidazol-5-yl-methanoyl)-amino]-propionic acid

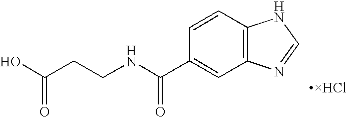

Example XI is prepared using the procedures described for the preparation of Example VI, substituting the appropriate amino acid starting material. This provides 11.6 mg of the product.

### Example XII

### 4-[(1-1H-Benzimidazol-5-yl-methanoyl)-amino]-butyric acid

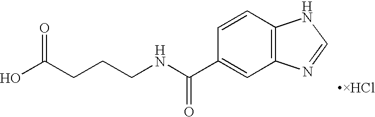

Example XII is prepared using the procedures described for the preparation of Example VI, substituting the appropriate amino acid starting material. This provides 15.3 mg of the product.

### Example XIII

### 3-{[(1-1H-Benzimidazol-5-yl-methanoyl)-amino]-methyl}-benzoic acid

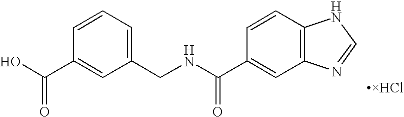

Example XIII is prepared using the procedures described for the preparation of Example VI, substituting the appropriate amino acid starting material. This provides 9.5 mg of the product.

### Example XIV

### (RS)-3-[(1-1H-Benzimidazol-5-yl-methanoyl)-amino]-3-phenyl-propionic acid

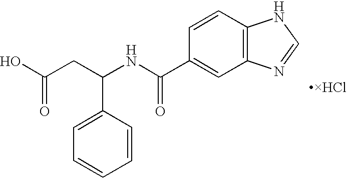

Example XIV is prepared using the procedures described for the preparation of Example VI, substituting the appropriate amino acid starting material. This provides 32.8 mg of the product.

### Examples Prepared from 2d

### Example XV

### (a¹S)-a-[[(2-amino-1H-benzimidazol-5-yl)carbonyl]amino]cyclo-hexanepropanoic acid hydrochloride

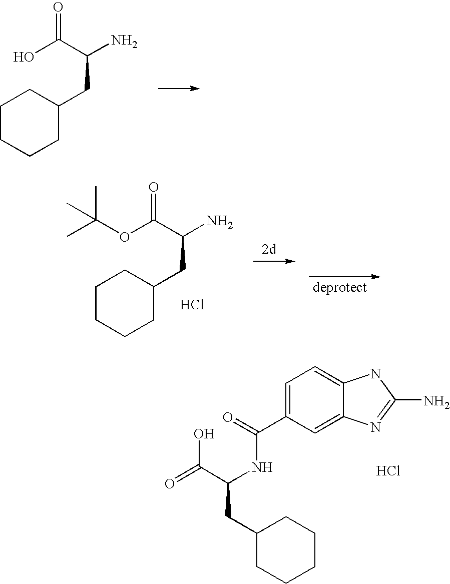

(a¹S)-a-aminocyclohexanepropanoic acid 1,1-dimethyl ester hydrochloride: (a¹S)-a-aminocyclohexanepropanoic acid (5.023 g, 24.2 mmol) is suspended in 25 mL of anhydrous 1,4-dioxane in a 200 mL Parr bottle. Concentrated sulfuric acid (3.05 mL, 57.2 mmol) is then added, and the resulting mixture is cooled in a dry-ice acetone bath. Approximately 25 grams of liquid isobutylene are condensed in a separate flask, and then added to the reaction mixture. The Parr bottle is stoppered, and allowed to warm to room temperature and stirred overnight to give a colorless solution. After cooling in a dry-ice acetone bath, the reaction bottle is opened and allowed to warm to room temperature. The mixture is basified with 1N NaOH, and then is extracted with ether (3×100 mL). The combined organics are washed with dil. NaHCO₃ solution. A solution of HCl in ether (1 M, 60 mL, 60 mmol) is added to precipitate the product, which is collected by filtration, rinsed with ether, and dried to give 4.285 g (67%) of the desired product.

(a¹S)-a-[[[2-[[(1,1-dimethylethoxy)carbonyl]amino]-1H-benzimidazol-5-yl]carbonyl]amino]cyclohexanepropanoic acid 1,1-dimethylethyl ester: To a suspension of (a¹S)-a-aminocyclohexanepropanoic acid 1,1-dimethyl ester hydrochloride (2.501 g, 9.481 mmol) in 100 mL of CH₂Cl₂ is added sequentially triethylamine (2.7 mL, 19.4 mmol), 2d (2.629 g, 9.481 mmol), 1-(3-dimethylaminopropyl)-3-ethylcarbodiimide hydrochloride (1.817 g, 9.50 mmol) and 1-hydroxybenzotriazole (1.150 g, 9.41 mmol) to give a brown suspension. The mixture is stirred at room temperature overnight, then filtered and concentrated. The residue is partitioned between ethyl acetate and 0.1M HCl. The organic layer is washed with dil. NaHCO₃ solution and brine, dried over sodium sulfate, and concentrated in vacuo to give 3.305 grams (71.6%) of the title compound as a yellowish tan foam.

(a¹S)-a-[[(2-amino-1H-benzimidazol-5-yl)carbonyl]amino]cyclohexanepropanoic acid hydrochloride: To a solution of (a¹S)-a-[[[2-[[(1,1-dimethylethoxy)carbonyl]amino]-1H-benzimidazol-5-yl]carbonyl]amino]cyclohexanepropanoic acid 1,1-dimethylethyl ester (2.288 g, 4.702 mmol) in 20 mL of anhydrous 1,4-dioxane is added 20 mL of 4M HCl-dioxane solution. A precipitate gradually forms. After stirring overnight, the mixture is concentrated, and the residue is partitioned between ethyl acetate and water to give an emulsion. The mixture is basified by addition of 1N NaOH, and the aqueous layer is lyophilized to give an impure product. This is dissolved in water and acidified to pH 6, precipitating the neutral product. This is collected by filtration. The resulting solid is treated with 20 mL of 1M HCl-diethyl ether and the mixture is stirred for 6 hours. The resulting solid is collected by filtration and rinsed with ether. The filtrate is dissolved in a minimal amount of water, filtered, and the filtrate lyophilized to give 1.421 g of product. The solution is concentrated and the residue is triturated with acetonitrile, filtered, and dried in vacuo at 60° C. to give 630 mg of Example XV as a solid.

### Example XVI

### (S)-2-{[1-(2-amino-1H-benzimidazol-5-yl)-methanoyl]-amino}-3-cyclohexylpropionic acid

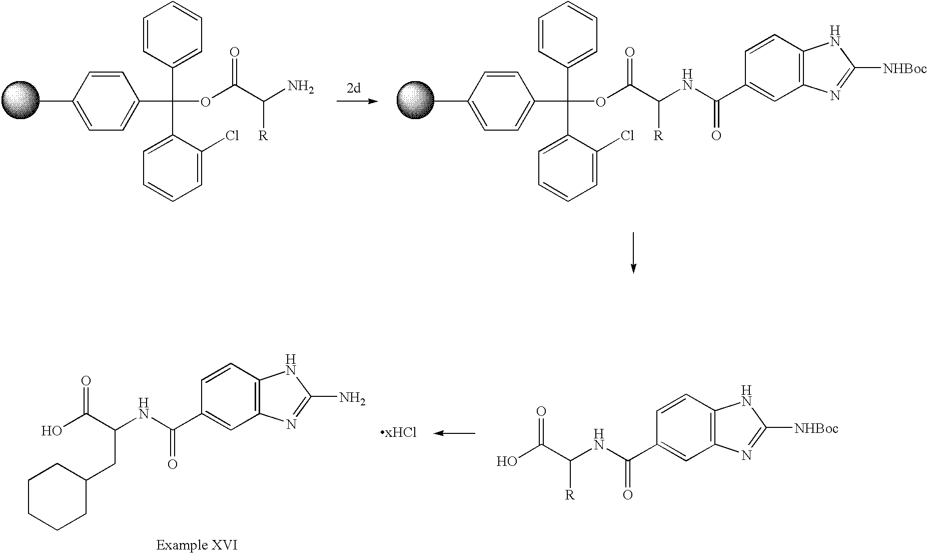

2d (1.1 g) is sonicated in a mixture of DCM (15 ml) and N-methylpyrrolidinone (5 ml) until it forms a fine suspension. Aliquots of this mixture (1 ml, >3 equivalents) are then added to the amino acid resin (60-80 mg, 1 equivalent), followed by diisopropylethylamine (84 ml, >6 equivalents). PyBroP (2.24 g) is dissolved in NMP (20 ml) and aliquots (1 ml, >3 equivalents) are added to the amino acid resin. The mixture is shaken overnight then filtered and the resin washed with DMF, DCM and MeOH. This sequence is repeated three times, then the resin is washed with DCM, MeOH and diethyl ether, this sequence being repeated twice. The degree of coupling is qualitatively assessed by Kaiser tests, and if required the above coupling procedure is repeated a second time.

The resin is treated with a mixture of AcOH, trifluoroethanol and DCM (1:1:8, 2 ml) for 4 hours. The filtrate is collected and the resin subjected to a second cleavage overnight. The filtrates are combined and concentrated in vacuo. Hexane is added and the mixture is concentrated again to azeotropically remove residual acetic acid. The crude products are purified by reverse phase chromatography (acetonitrile/water gradient) to afford (S)-2-{[1-(2-tert-butoxycarbonylamino-1H-benzimidazol-5-yl)-methanoyl]-amino}-3-cyclohexylpropionic acid (13.9 mg) and (S)-2-{[1-(2-tert-butoxycarbonylamino-1H-benzimidazol-5-yl)-methanoyl]-amino}-3-(4-chlorophenyl)-propionic acid (13.8 mg).

The BOC protected compounds are treated with 4M HCl in dioxane (1 ml) for 24 h. Concentration gives 17.3 mg of Example XVI.

### Example VII

### (2S)-2-[(N-BOC-2-aminobenzimidazol-5-yl)carbonylamino]-3-(p-chlorophenyl)propanoic acid hydrochloride

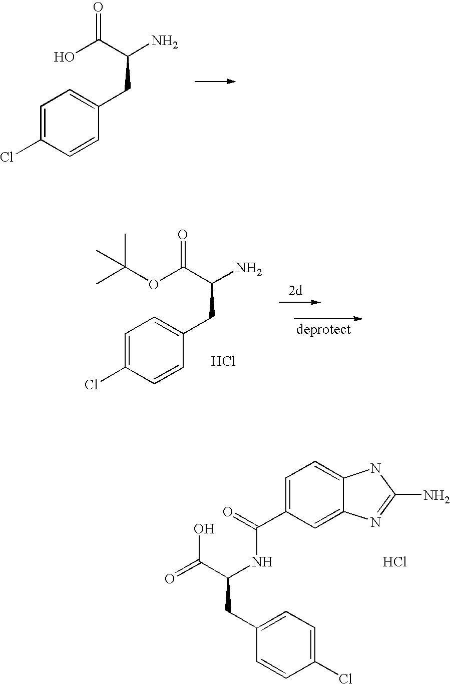

p-Chloro-L-phenylalanine t-butyl ester hydrochloride: p-Chloro-L-phenylalanine (5.10 g, 25.5 mmol) is suspended in anhydrous 1,4-dioxane (25 mL) in a Parr bottle and concentrated sulfuric acid (3.05 mL) is added to give a pale yellow solution with some insoluble chunks. The reaction mixture is cooled to −78° C. Isobutylene (29.4 g) is condensed and added to the Parr bottle. The bottle is sealed and then allowed to stir at room temperature overnight. The resulting pale yellow solution is re-cooled before opening, but then allowed to warm to room temperature before adding 1N sodium hydroxide to bring to pH 10. Excess isobutylene is allowed to evaporate and the reaction mixture is then extracted with diethyl ether (3×100 mL). The combined organic layers arre washed with dilute sodium bicarbonate solution, dried over MgSO₄, and concentrated to about 200 mL. To this concentrate is 1M HCl in ether (50 mL) with stirring. After several minutes, the white precipitate which is formed, is filtered and rinsed with ether to give 5.61 g (75%) of the desired product as a solid.

t-Butyl (2S)-2-[(N-BOC-2-aminobenzimidazol-5-yl)carbonylamino]-3-(p-chlorophenyl)propanoate: p-Chloro-L-phenylalanine t-butyl ester hydrochloride (2.50 g, 8.56 mmol) is suspended in dichloromethane (100 mL). Triethylamine (2.4 mL, 17.22 mmol) is added to give a solution. 2d (2.37 g, 8.56 mmol), 1-(3-dimethylaminopropyl-3-ethylcarbodiimide hydrochloride (1.64 g, 8.57 mmol), and 1-hydroxybenzotriazole (1.15 g, 8.55 mmol) are added to the reaction mixture to give a brown suspension. After stirring 3 hours at room temperature, tlc indicates that the reaction is complete. The reaction mixture is partitioned between 0.1N HCl and dichloromethane and resulting emulsion is filtered through a pad of Celite® to help separate the layers. The organic layer is washed with dilute sodium bicarbonate solution and brine and dried over sodium sulfate. Concentration gave a foamy solid which was applied to a column of silica gel in 5% methanol/dichloromethane. Concentration of appropriate fractions gave 3.27 g (74%) and the desired product as a foamy solid.

(2S)-2-[(N-BOC-2-aminobenzimidazol-5-yl)carbonylamino]-3-(p-chlorophenyl)propanoic acid hydrochloride: t-Butyl (2S)-2-[(N-BOC-2-aminobenzimidazol-5-yl)carbonylamino]-3-(p-chlorophenyl)propanoate (2.21 g, 4.28 mmol) is dissolved in anhydrous 1,4-dioxane (20 mL) under nitrogen atmosphere. To the resulting light brown solution is added 4M HCl in 1,4 dioxane (16 mL) and the reaction mixture is then allowed to stir at room temperature overnight as a precipitate slowly forms. The reaction mixture is concentrated and partitioned between ethyl acetate and water. The aqueous layer is lyophilized and the resulting solid is recrystallized from aqueous acetonitrile to give 580 mg (34%) of the desired product after drying in a vacuum oven for 3 days.

### Example VIII

### (S)-2-{[1-(2-amino-1H-benzimidazol-5-yl)-methanoyl]-amino}-3-(4-chlorophenyl)-propionic acid

The procedures used to prepare Example XVI are used to prepare 16.4 mg of Example XVIII, substituting the appropriate amino acid starting material.

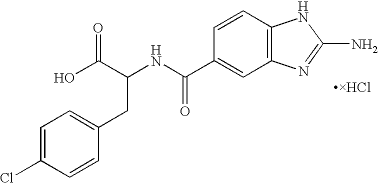

### Example XIX

### 3-({[1-(2-Amino-1H-benzimidazol-5-yl)-methanoyl]-amino}-methyl)-benzoic acid

The procedures used to prepare Example XVI are used to prepare 15.7 mg of Example XIX, substituting the appropriate amino acid starting material.

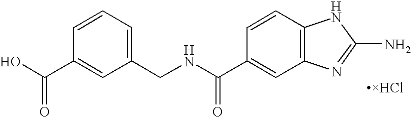

### Example XX

### 3-{[1-(2-amino-1H-benzimidazol-5-yl)-methanoyl]-amino}-propionic acid

The procedures used to prepare Example XVI are used to prepare 14.7 mg of Example XX, substituting the appropriate amino acid starting material.

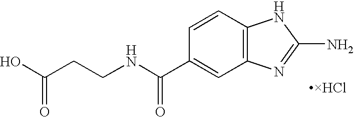

### Example XXI

### (S)-2-{[1-(2-Amino-1H-benzimidazol-5-yl)-methanoyl]-amino}-3-thiophen-2-yl-propionic acid

The procedures used to prepare Example XVI are used to prepare 12.4 mg of Example XXI, substituting the appropriate amino acid starting material.

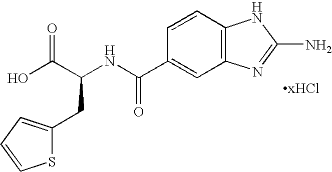

### Examples Prepared from 2e

### Example XXII

### 3-(4-Chloro-phenyl)-2-{[1-(2-methyl-3H-benzimidazol-5-yl)-methanoyl]-amino}-propionic acid

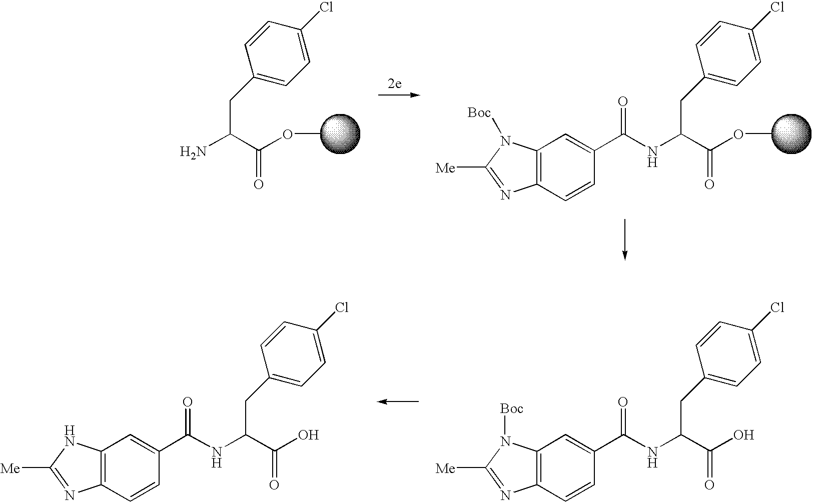

The 2-chlorotrityl chloride resin loaded with the p-Cl-L-Phe-OH amino acid is suspended in 1-methyl-2-pyrrolidinone (10 mL) and DIPEA (6 eq). 2-Methyl-benzimidazole-1,6-dicarboxylic acid 1-tert-butyl ester (3 eq) and PyBroP (3 eq) are added and the reaction agitated with a shaker for 24 hours. The resin is filtered and washed three times with DCM, twice with DMF, twice with DCM and three times with diethyl ether. It is then dried in vacuo. The resin is subjected to a Kaiser test.

Cleavage from the resin is performed with 10% TFE/10% AcOH/80% DCM. The reaction is shaken for 2 hours. The mixture is concentrated in vacuo. Hexane (15 times volume) is added and evaporated to remove AcOH as an azeotrope.

6-[1-Carboxy-2-(4-chlorophenyl)-ethylcarbamoyl]-2-methyl-benzimidazole-1-carboxylic acid tert-butyl ester is treated with 4M HCl in 1,4 dioxane (10 ml). The reaction is maintained at room temperature overnight. The mixture is concentrated to afford Example XXII.

### Example XXIII

### (R)-2-{[1-(2-Methyl-3H-benzimidazol-5-yl)-methanoyl]-amino}-3-naphthalen-2-yl-propionic acid

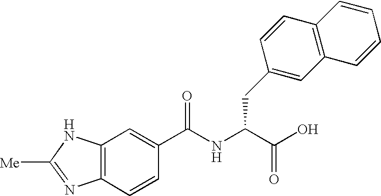

This compound is prepared using the procedures described for the preparation of Example XXII, substituting the appropriate amino acid starting material.

### Example XXIV

### (R)-3-Cyclohexyl-2-{[1-(2-methyl-3H-benzoimidazol-5-yl)-methanoyl]-amino}-propionic acid

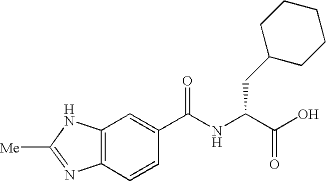

This compound is prepared using the procedures described for the preparation of Example XXII, substituting the appropriate amino acid starting material.

### Example XXV

### 3-({[1-(2-Methyl-3H-benzimidazol-5-yl)-methanoyl]-amino}-methyl)-benzoic acid

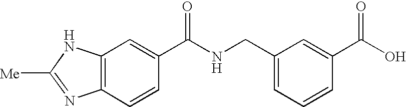

This compound is prepared using the procedures described for the preparation of Example XXII, substituting the appropriate amino acid starting material.

### Examples Prepared from 2f

### Example XXVI

### 2-{[1-(3-Methyl-3H-benzimidazol-5-yl)-methanoyl]-amino}-3-p-tolyl-propionic acid

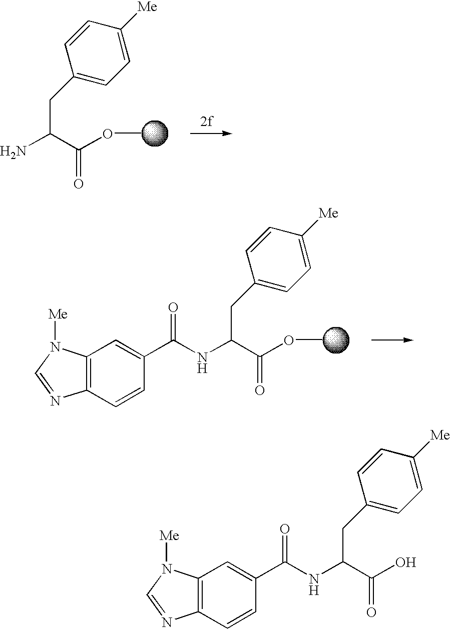

The 2-chlorotrityl chloride resin loaded with p-Me-L-Phe-OH amino acid is suspended in 1-methyl-2-pyrrolidinone (10 mL) and DIPEA (6 eq). 3-Methyl-3H-benzimidazole-5-carboxylic acid (3 eq) and PyBroP (3 eq) are added and the reaction is agitated with a shaker for 24 hours. The resin is filtered and washed three times with DCM, twice with DMF, twice with DCM and three times with diethyl ether. It is then dried in vacuo.

Cleavage from the resin is performed with 10% TFE/10% AcOH/80% DCM. The reaction is left to shake for 2 hours. The mixture is concentrated in vacuo. Hexane (15 times volume) is added and evaporated to remove AcOH as an azeotrope.

### Example XXVII

### 3-(4-Chlorophenyl)-2-{[1-(3-methyl-3H-benzimidazol-5-yl)-methanoyl]-amino}-propionic acid

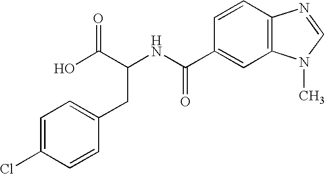

This compound is prepared using the procedures described for the preparation of Example XXVI, substituting the appropriate amino acid starting material.

### Examples Prepared from 2g

### Example XXVIII

### 2-(2-methoxybenzimidazol-6-ylcarbonylamino)-3-phenylpropanoic acid

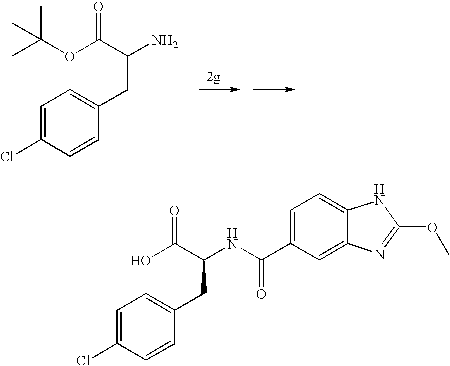

The amino acid (452 mmol) and 2g (86 mg, 452 mmol) are dissolved in dry NMP (5 ml). Diisopropylethylamine (233 mg, 1.808 mmol) is added followed by PyBroP (253 mg, 542 mmol) in a single portion. The reaction is stirred overnight and then diluted with EtOAc (50 ml). The organics are washed with water, saturated NaHCO₃, 10% citric acid, and brine (10 ml each), and then dried over MgSO₄, filtered, and concentrated in vacuo. The residue is purified by passing the crude product through a plug of silica, eluting with 50% EtOAc/hexane followed by 80% EtOAc/hexane to yield the intermediate ester.

The ^{t}Bu ester (72 mg, 182 mmol) is dissolved in 4M HCl/dioxane (2 ml) and stirred overnight. The residue is concentrated and taken up in THF/H2O (1.5 ml each) and LiOH.H₂O (150 mg, 3.57 mmol) is added. After 6 hrs the reaction is taken to pH 1 with 1M HCl and extracted with EtOAc (30 ml). The EtOAc is dried over MgSO₄, filtered, and concentrated to yield Example XXVIII.

### Examples Prepared from 2h

### Example XXIX

### 2-[(1-Benzothiazol-6-yl-methanoyl)-amino]-3-phenyl-propionic acid

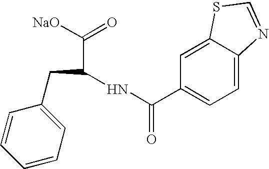

Benzothiazole-6-carboxylic acid (2h, 258 mg) is dissolved in dichloromethane (10 ml), and treated with 1-(3-dimethylaminopropyl)-3-ethylcarbodiimide hydrochloride (345 mg). The reaction is stirred for 10 minutes. 1-Hydroxybenzotriazole (244 mg) is added and the mixture is stirred for a further 10 minutes. L-Phenylalanine t-butyl ester hydrochloride (200 mg) is added and the reaction maintained at room temperature for 72 hours. The mixture is concentrated, the residue taken up in ethyl acetate and washed with water, sodium bicarbonate, and brine. The organic phase is dried (MgSO₄), concentrated, and then purified by chromatography on silica (10% diethyl ether/DCM). This afforded the ester intermediate as a solid (60 mg).

2-[(1-Benzothiazol-6-yl-methanoyl)-amino]-3-phenyl-propionic acid tert-butyl ester (60 mg), is treated with 4M HCl in 1,4 dioxane (10 ml), and the reaction maintained at room temperature overnight. The mixture is concentrated to afford Example XXIX(18 mg).

### Examples Prepared from 2i

### Example XXX

### (S)-2-{[1-(2-hydroxybenzoxazol-5-yl)-methanoyl]-amino}-3-(1H-imidazol-4-yl)-propionic acid

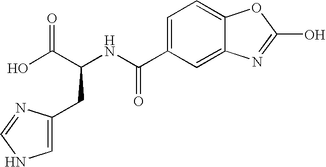

The resin bound amino acid (100 mg, ˜100 mmol) is suspended in NMP (2 ml), and diisopropylethylamine (77 mg, 600 mmol) is added followed by 2i (300 mmol) and PyBroP (139 mg, 300 mmol). The reaction is shaken for 72 hrs then, and then is washed with MeOH, DCM (twice), MeOH, THF (twice), MeOH, DCM, and MeOH (twice), and then is dried under suction. The bromophenol blue test indicates that the reaction is complete.

The resin is then treated with 2M HCl dioxane (2 ml) for 1 hr. The resin mixture is filtered and washed with dioxane (twice) and water, and then the aqueous dioxane solution is concentrated in vacuo to yield the product.

### Example XXXI

### 3-(4-chlorophenyl)-3-{[1-(2-hydroxybenzoxazol-5-yl)-methanoyl]-amino}-propionic acid

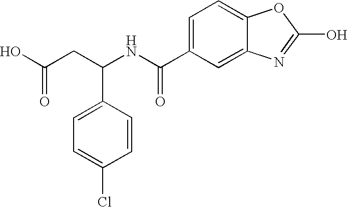

Example XXXI is prepared using the procedure described for the preparation of Example XXX, substituting the appropriate amino acid.

### Example XXXII

### (S)-3-cyclohexyl-2-{[1-(2-hydroxy-benzoxazol-5-yl)-methanoyl]-amino}-propionic acid

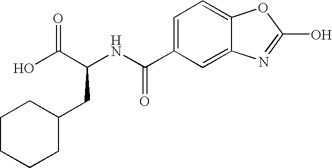

Example XXXII is prepared using the procedure described for the preparation of Example XXX, substituting the appropriate amino acid.

### Example XXXIII

### (1R,2S)-2-{[1-(2-hydroxy-benzoxazol-5-yl)methanoyl]-amino}-cyclohexanecarboxylic acid

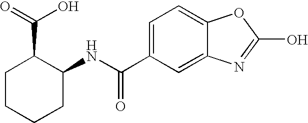

Example XXXIII is prepared using the procedure described for the preparation of Example XXX, substituting the appropriate amino acid.

### Example XXXIV

### (S)-{[1-(2-hydroxy-benzoxazol-5-yl)-methanoyl]-amino}-phenylacetic acid

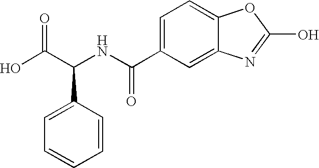

Example XXXIV is prepared using the procedure described for the preparation of Example XXX, substituting the appropriate amino acid.

### Example XXXV

### 3-{[1-(2-hydroxy-benzoxazol-5-yl)-methanoyl]-amino}-propionic acid

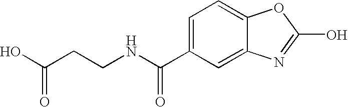

Example XXXV is prepared using the procedure described for the preparation of Example XXX, substituting the appropriate amino acid.

### Examples Prepared from 2j

### Example XXXVI

### (S)-2-{[1-(2-Aminobenzoxazol-5-yl)-methanoyl]-amino}-3-phenylpropionic acid tert-butyl ester

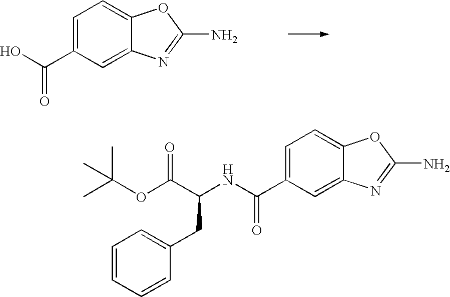

H Phe O^{t}Bu.HCl (100 mg, 452 mmol) is dissolved in NMP (5 ml), and 2j (140 mg, 542 mmol) is added followed by diisopropylethylamine (233 mg, 1.808 mmol) and PyBroP (295 mg, 632 mmol). The reaction is stirred overnight and then diluted with EtOAc (50 ml). The organics are washed with water (3×10 ml), saturated NaHCO₃ soln. (10 ml), 10% citric acid (10 ml), and brine (10 ml), and then are dried over MgSO₄, filtered, and concentrated. Additional washing may be necessary to remove all NMP. This provides 185 mg of Example XXXVI.

### Example XXXVII

### (S)-2-({[1-(2-Amino-benzoxazol-5-yl)-methanoyl]-amino}-3-phenylpropionic acid

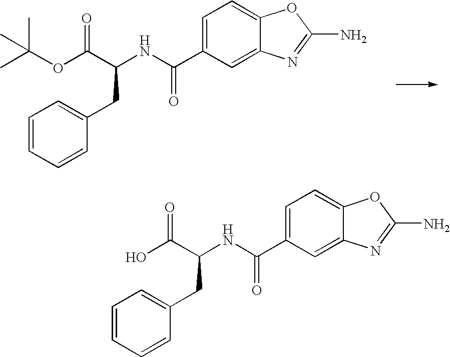

The ^{t}Bu ester is dissolved in 4M HCl dioxane and stirred overnight. Concentration of the mixture yields the product.

### Additional Examples

### Example XXXVIII

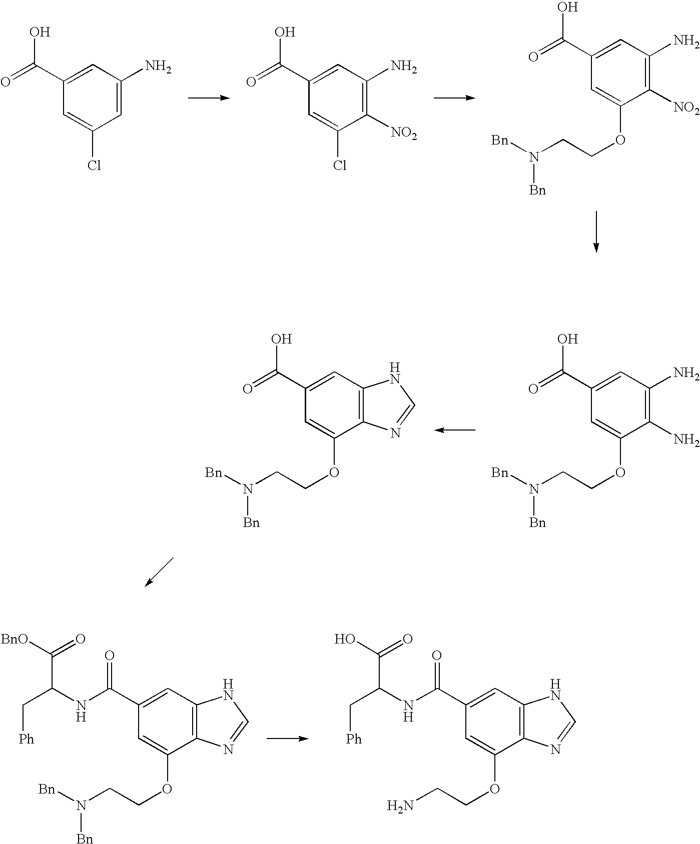

Standard aromatic ring chemistry is used to prepare the known starting material, 3-amino-5-chloro-benzoic acid (CAS #21961-30-8). This compound is nitrated under standard conditions, and the desired 4-nitro regioisomer is isolated via silica gel chromatography.

The 5-chloro group is displaced with a nucleophile, in this case 2-(N-dibenzyl)-aminoethanol, in the presence of a base. The nitro group is reduced to give the diamine intermediate, which is then cyclized to the benzimidazole under standard conditions. Coupling to the benzyl ester of phenylalanine is achieved via standard carbodiimide chemistry described herein. Global hydrogenolysis allows cleavage of the three benzyl groups to yield Example XXVIII.

### Example XLIX

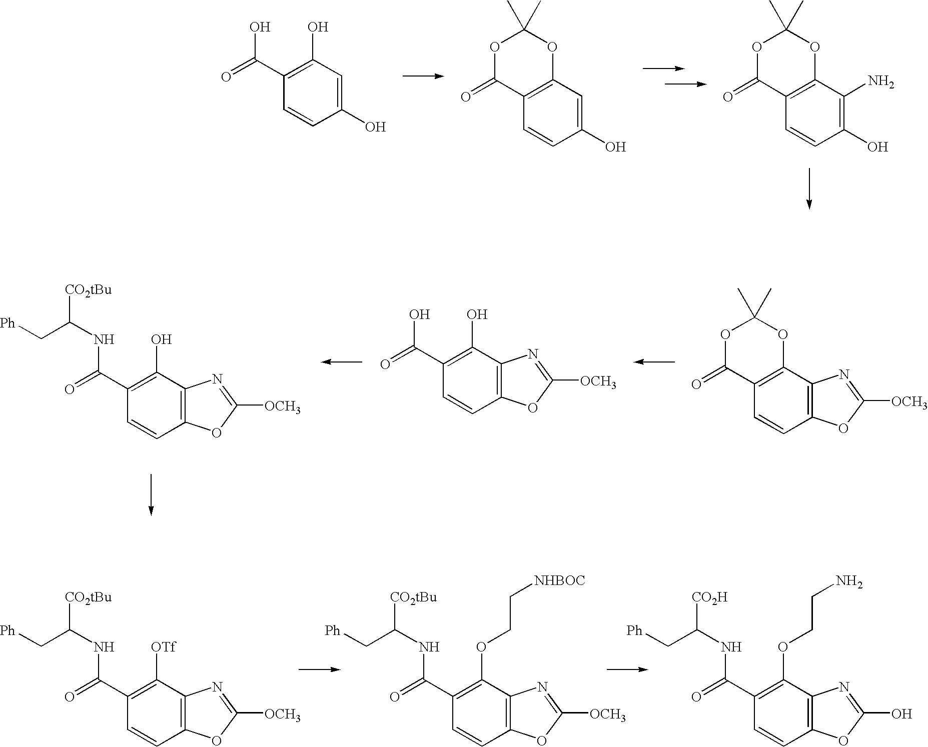

2,4-dihydroxyphenol is stirred in trifluoroacetic acid, and this mixture is treated with acetone and trifluoroacetic anhydride. After stirring until completion, the mixture is partitioned between neutral buffer and ethyl acetate. The organics are dried and concentrated to provide the dioxenone intermediate, which may be purified via silica gel chromatography. The aromatic amine is formed via standard nitration procedure, followed by reduction with a reagent system such as iron/HOAc. The desired regioisomer may be isolated via silica gel chromatography.

Formation of the 2-methoxybenzoxazole is achieved by treatment with tetramethyl orthocarbonate and acetic acid. The reaction may be heated to about 100° C. Upon completion, the reaction is diluted with EtOAc and saturated NaHCO₃. The organics are dried over MgSO₄, filtered, and concentrated. The product is purified by column chromatography. The dioxenone is then hydrolyzed under standard basic conditions, such as KOH in aqueous dioxane, to provide the 2-methoxy-4-hydroxy-5-carboxybenzoxazole intermediate.

Coupling to the t-butyl ester of phenylalanine is achieved via standard carbodiimide chemistry described herein. The aromatic hydroxyl group is converted to the triflate using triflic anhydride and triethylamine in dichloromethane. The triflate is displaced with a nucleophile, in this case 2-(N-t-butoxycarbonyl)-aminoethanol. Global deprotection under acidic conditions allows cleavage of the t-butyl ester, the BOC group, and the methyl group at the 2-position to yield the 4-substituted (R9) 2-hydroxybenzoxazole, Example XLIX.

### Example XL

### 3H-Benzimidazole-5-carboxylic acid [(S)-2-phenyl-1-(1H-tetrazol-5-yl)-ethyl]-amide

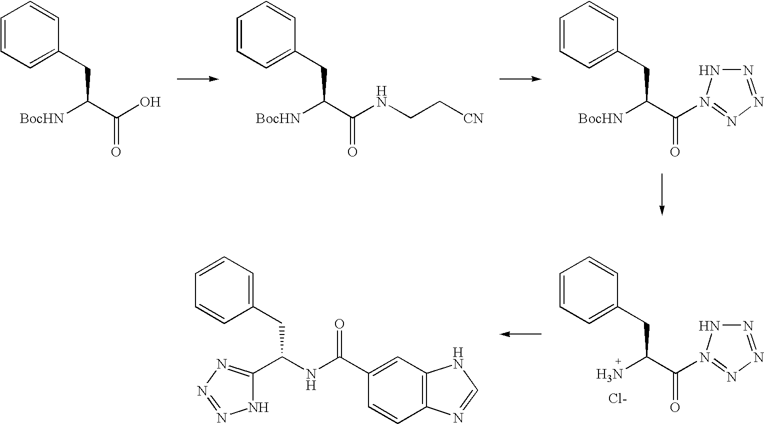

[(S)-1-(2-Cyano-ethylcarbamoyl)-2-phenyl-ethyl]-carbamic acid tert-butyl ester: BOC Phe OH (1 g, 3.77 mmol) is suspended in dry DCM (20 ml) and DIPEA (1.07 g, 8.3 mmol) is added followed by HOBT (560 mg, 4.15 mmol) and 3 aminopropionitrile (290 mg, 4.15 mmol). EDC (795 mg, 4.15 mmol) is added and the reaction is stirred overnight. The reaction is then diluted with DCM (100 ml) and washed with 10% citric acid (30 ml), saturated NaHCO₃ (30 ml), and brine (30 ml), then the organics dried over MgSO₄, filtered, and concentrated to yield a white solid.

[(S)-2-Phenyl-1-(1H-tetrazol-5-yl)-ethyl]-carbamic acid tert-butyl ester:

The cyano intermediate (1 g, 3.15 mmol) is dissolved in dry THF (32 ml), and PPh₃ (1.652 g, 6.3 mmol) is added followed by diethylazodicarboxylate (DEAD) (1.097 g, 6.3 mmol) in a dropwise fashion to keep the exothermic reaction from overheating. After 10 minutes, trimethylsilylazide (TMSN₃) (726 mg, 6.3 mmol) is added slowly and the reaction is stirred for 4 days. The reaction is concentrated carefully and then redissolved in THF (10 ml). 1M NaOH (6 ml, 2 eq) is added and the reaction is stirred overnight. The reaction is diluted with water (75 ml) and 1M NaOH (30 ml), and washed with Et₂O (3×50 ml). The aqueous phase is acidified to pH 2 with conc. HCl and then cooled in an ice bath. After about 1 hr the solid precipitate is collected by filtration and dried under vacuum to yield a white solid.

(S)-2-Phenyl-1-(1H-tetrazol-5-yl)-ethyl-ammonium hydrochloride: The tetrazole (300 mg, 1.037 mmol) is suspended in 4M HCl in dioxane (4 ml). EtOAc (4 ml) and water (1 ml) are added to promote dissolution. The now clear solution is stirred for 24 hrs and then water (2 ml) is added and the reaction concentrated on a freeze drier to yield a white solid.

3H-Benzimidazole-5-carboxylic acid [(S)-2-phenyl-1-(1H-tetrazol-5-yl)-ethyl]-amide: The amine hydrochloride (100 mg, 443 mmol) is suspended in dry DCM (1 ml), and is treated with benzimidazole 5 carboxylic acid (86 mg, 531 mmol) and DIPEA (172 mg, 1.33 mmol). O-(7-azabenzotriazol-1-yl)-N,N,N′N′-tetramethyluronium hexafluorophosphate (HATU) (185 mg, 487 mmol) is added and the reaction is stirred for 3 days. The reaction is diluted with EtOAc (50 ml) and washed with 10% citric acid (20 ml), saturated NaHCO₃ (20 ml), and brine (20 ml), and then dried over MgSO₄, filtered, and concentrated. The product is purified by preparative HPLC on a Phenomenex C8 column, eluting with water/MeCN (0.1% formic acid).

### Example XLI

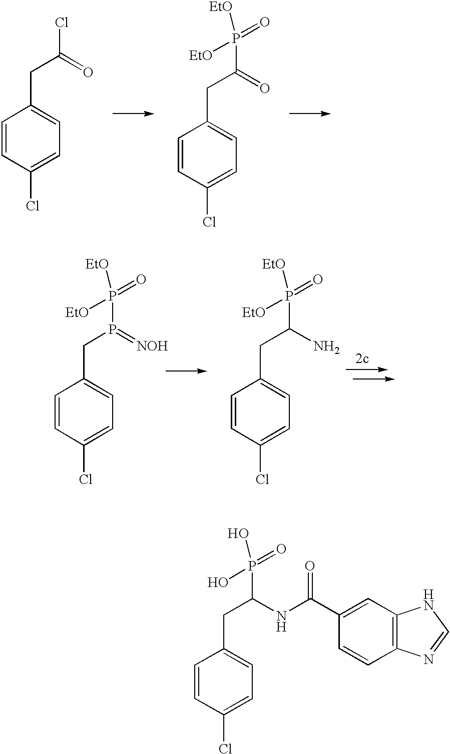

The acid chloride starting material is converted to the corresponding keto-phosphonate diethyl ester via standard conditions using triethylphosphite. This material is converted to the oxime with hydroxylamine hydrochloride and pyridine in ethanol. The oxime is reduced with sodium borohydride in the presence of a transition metal (MoO₃) to give the amine, which is coupled to BOC-protected 5-carboxybenzimidazole (2c) to yield the protected intermediate. Removal of the BOC group is achieved with trifluoroacetic acid, and the phosphonate ester is converted to the desired phosphonic acid (Example XLI) with trimethylsilyl bromide in acetonitrile.

### Example XLII

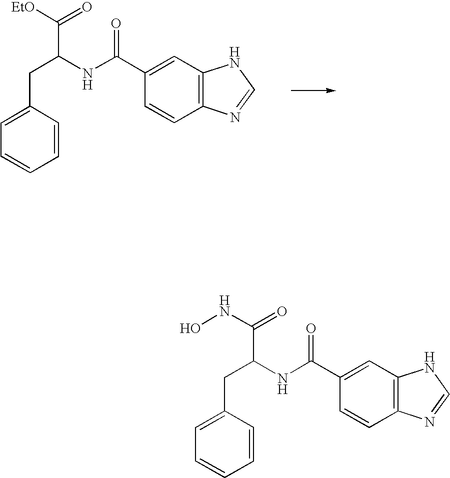

The chemistry used to prepare Example I is used to prepare Example ppp, except that the ethyl ester of phenylalanine is used in place of the benzyl ester. This yields the starting material in the scheme shown above. This compound is converted to the corresponding hydroxamic acid (Example XLII) under standard conditions.

### Example XLIII

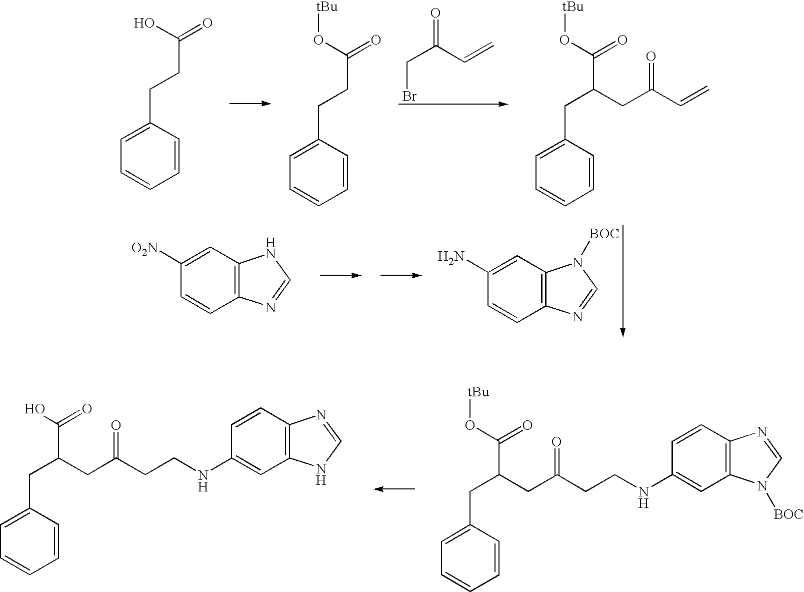

3-Phenylpropionic acid is converted to the corresponding t-butyl ester via reaction with isobutylene. This material is deprotonated with lithium diisopropylamide (LDA) and the resulting nucleophile is treated with 1-bromo-3-butene-2-one (CAS #155622-69-8). The desired enone intermediate may be purified by silica gel chromatography.

5-Nitrobenzimidazole is protected with the BOC group under standard conditions, and the protected product is reduced under standard conditions to give the BOC-protected 5-aminobenzimidazole shown in the scheme. This material is added to the enone intermediate in a 1,4-fashion using standard conditions reported in the literature. The resulting adduct is deprotected under acidic conditions to give Example XLIII.

### Example XLIV

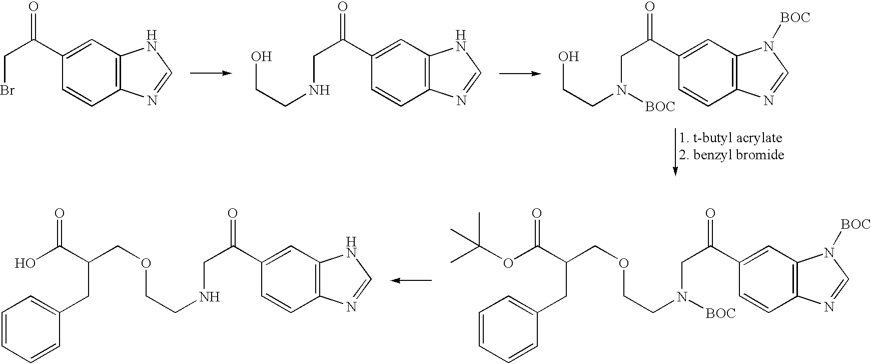

5-(Bromoacetyl)benzimidazole (CAS #124663-08-7) is treated with 2-aminoethanol to give the secondary amine product, which may be purified by silica gel chromatography. This material is doubly protected with the BOC group under standard conditions. The resulting primary alcohol intermediate is treated with base to form the corresponding alkoxide, which is added in a 1,4-fashion to t-butyl acrylate. The nucleophilic intermediate generated via this process is trapped with benzyl bromide to give the triply protected intermediate shown in the scheme. Global deprotection under acidic conditions provides Example XLIV.

Compositions

Compositions of this invention comprise a safe and effective amount of the above described compounds, and a pharmaceutically-acceptable carrier. As used herein, “safe and effective amount” means an amount of a compound sufficient to significantly induce a positive modification in the condition to be treated, but low enough to avoid serious side effects (at a reasonable benefit/risk ratio), within the scope of sound medical judgment. A safe and effective amount of a compound will vary with the particular condition being treated, the age and physical condition of the patient being treated, the severity of the condition, the duration of the treatment, the nature of concurrent therapy, the particular pharmaceutically-acceptable carrier utilized, and like factors within the knowledge and expertise of the attending physician.

In addition to the compound, the compositions of this invention contain a pharmaceutically-acceptable carrier. The term “pharmaceutically-acceptable carrier”, as used herein, means one or more compatible solid or liquid filler diluents or encapsulating substances which are suitable for administration to a subject. The term “compatible”, as used herein, means that the components of the composition are capable of being commingled with the compound, and with each other, in a manner such that there is no interaction which would substantially reduce the pharmaceutical efficacy of the composition under ordinary use situations. Pharmaceutically-acceptable carriers must, of course, be of sufficiently high purity and sufficiently low toxicity to render them suitable for administration to the subject being treated.

Some examples of pharmaceutically-acceptable carriers or components thereof are sugars, such as lactose, glucose, and sucrose; starches, such as cornstarch and potato starch; cellulose and its derivatives, such as sodium carboxymethyl cellulose, ethyl cellulose, cellulose acetate; powdered tragacanth; malt; gelatin; talc; solid lubricants, such as stearic acid, magnesium stearate; or calcium sulfate; vegetable oils, such as peanut oil, cottonseed oil, sesame oil, olive oil, corn oil, and oil of theobroma; polyols such as propylene glycol, glycerin, sorbitol, mannitol, and polyethylene glycol; alginic acid; emulsifiers, such as the Tweens®; wetting agents such as sodium lauryl sulfate; coloring agents; flavoring agents; excipients; tableting agents; stabilizers; antioxidants; preservatives; pyrogen-free water; isotonic saline; and phosphate buffer solutions.

The choice of a pharmaceutically-acceptable carrier to be used in conjunction with a compound is basically determined by the way the compound is to be administered. The compounds and compositions of the present invention may be administered systemically. Routes of administration include topical or transdermal (patch, ointment, cream, powder, etc.); oral; parenteral, including subcutaneous, intramuscular, or intravenous injection; topical; rectal; colonic; intraperitoneal; intraoccular; sublingual; buccal; inhalation; and/or intranasal. The preferred route of administration is parenteral, especially intravenous injection on a daily or as needed basis.

The appropriate amount of the compound to be used may be determined by routine experimentation with animal models. Such models include, but are not limited to the ferret, canine, and non human primate models. Generally, an amount between 0.01 μg/kg to 100 mg/kg of body weight per day is administered dependent on the potency of the compound or compositions used.

Preferred unit dosage forms for injection include sterile solutions of water, physiological saline, or mixtures thereof. Parenteral unit dosage form compositions may be in the form of solutions ready for injection or dry (e.g. lyophilized) compositions which are reconstituted with water or saline solutions prior to injection. The pH of said solutions should be adjusted to about 7.4. Suitable carriers for injection or surgical implants include hydrogels, controlled- or sustained release devises, polylactic acid, and collagen matrices. Other suitable carriers for injection include dextrose, mannitol, lactose, lecithin, albumin, sodium glutamate, and the like.

Suitable pharmaceutically-acceptable carriers for topical application include those suited for use in lotions, creams, gels, and the like. If the compound is to be administered perorally, the preferred unit dosage form is tablets, capsules, elixirs, and the like. If the compound is to be administered rectally, the preferred unit dosage form is suppositories, and the like. The pharmaceutically-acceptable carriers suitable for the preparation of unit dosage forms for oral, rectal, topical, and perenteral administration are well-known in the art. These carriers are described in Remington's Pharmaceutical Sciences, Mack Publishing Co. (19th edit. 1995); Modern Pharmaceutics, Vol. 7, Chapters 9 & 10, Banker & Rhodes (1979); Lieberman, et al, Pharmaceutical Dosage Forms: Tablets (1981); and Ansel, Introduction to Pharmaceutical Dosage Forms, 2d (1976). Their selection will depend on secondary considerations like taste, cost, and shelf stability, which are not critical for the purposes of the subject invention, and can be made without difficulty by those skilled in the art.

Methods of Use

The compounds of the present invention are useful in treating any disease state where the prevention of tissue damage due to an event is needed. Events leading to tissue damage include aneurysm repair, coronary bypass, transplant surgery, traumatic hemorrhage, organ ischemia due to hypoperfusion, sepsis, etc. While applicants do not wish to be bound by any theory or mode of activity, applicants believe that the compounds of this invention, at least in part, bind to integrins on neutrophils such as Mac-1 to inhibit or reduce neutrophil activity, and/or decrease neutrophil migration. Thus these compounds can be used to treat conditions which involve undesirable or abnormal inflammatory responses including, but not limited to, chronic inflammatory lung diseases (such as emphysema, bronchitis, adult respiratory distress syndrome [ARDS], asthma); ischemia-reperfusion injury (such as ischemia-reperfusion injury following events like myocardial infarction, coronary artery bypass grafting, angioplasty, angina, shock, stroke, perinatal asphyxia, surgery, replantation, renal failure, peripheral vascular disease; valve disease; tissue and organ transplantation, acute and chronic allograft rejection, vasculitis); arthritis (such as rheumatoid arthritis); inflammatory bowel disease (such as ulcerative colitis, Crohn's disease); hepatitis; pancreatitis; allergy; gout; radiation; ulcer; fibrosis; migraine; and inflammatory skin diseases (such as burns, frostbite, psoriasis, sunburn, utacaria, etc.). In addition, the compounds are potentially useful for treatment of cardiovascular disease or vascular disease (such as myocardial ischemia, angina, cardiac arrhythmia, heart failure, hypertension, peripheral vascular disease, valve disease, vasculitis, sepsis, etc.).

The dosage range of the compound for systemic administration is from about 0.01 to about 1000 μg/kg body weight, preferably from about 0.1 to about 100 μg/kg per body weight, most preferably from about 1 to about 50 μg/kg body weight per day. The transdermal dosages will be designed to attain similar serum or plasma levels, based upon techniques known to those skilled in the art of pharmacokinetics and transdermal formulations. Plasma levels for systemic administration are expected to be in the range of 0.01 to 100 ng/ml, more preferably from 0.05 to 50 ng/ml, and most preferably from 0.1 to 10 ng/ml. While these dosages are based upon a daily administration rate, weekly or monthly accumulated dosages may also be used to calculate the clinical requirements.

Dosages may be varied based on the patient being treated, the condition being treated, the severity of the condition being treated, the route of administration, etc. to achieve the desired effect.

In order to determine and assess pharmacological activity, testing of the subject compounds is carried out using various assays known to those skilled in the art. For example, the neutrophil inhibitory activity of the subject compounds can be conveniently demonstrated using an in vitro assay designed to test the ability of the subject compounds to reduce migration of the neutrophils across the endothelial cell wall. An example of such an assay is the transendothelial migration assay (TEM), measuring the migration of neutrophils across an endothelial monolayer as follows:

Preparation of Primary Endothelial Cells (HUVEC)

Initially frozen primary human umbilical vein endothelial cells (HUVEC) (pooled, Cat. No. CC2519, Clonetics Biowhittaker) are subcultured in complete endothelial cell basal medium (EGM) (Cat. No. CC4133 Clonetics) in a T-75 culture flask (Cat. No. 24599032, Corning). ¹ On the day prior to confluency of the HUVEC, each appropriate well of a 96 well tissue culture plate (flat bottom, Cat. No. 3596, Costar) is coated with 50 μl of collagen (8:1:5;

Vitrogen [Cat. No. PC701, Collagen Biomedical], Medium 199-10X [Cat. No. 11181-039, Gibco Life Technologies], 0.1 N NaOH² [Cat. No. 5636-02, JTS Baker]) and allowed to gel by incubating at 37° C. for 30 to 60 minutes. When the gel is firm it is overlaid with an equal volume of Medium 199 (1X) (Cat. No. 12350-039, Gibco Life Technologies) and then equilibrated overnight in the tissue culture incubator. The day in which the HUVEC are to be subcultured from the culture flask, the Medium 199 is aspirated from the plates and a 50 μl aliquot of fibronectin³ (50 μg/ml) (Fibronectin Human 1 mg, Cat. No. 4008, Collaborative Biomedical Products) is coated on the surface of the gel, the plates are then placed in a CO₂ incubator at 37° C. for approximately 15 minutes. During this 15 minutes the confluent monolayer of endothelial cells in the T75 flask is detached using a trypsin solution (EDTA 1× Solution, Cat. No. T-3924 Sigma). The medium from T75 flask with confluent layer of HUVEC is aspirated. The layer of cells in flask is rinsed with 10 ml of warm PBS. The PBS is aspirated from flask. 5 ml of warm trypsin solution is added to T75 flask. This is incubated for 3 minutes at 37° C. in a CO₂ incubator. Following incubation the side of flask is gently tapped to dislodge cells. 7 ml of EGM medium is added to flask with cells and trypsin. The cell mixture is removed from flask and placed in a 15 ml centrifugation tube. The cells are centrifuged at 980 rpm for 10 minutes at room temperature. Following centrifugation, medium is removed from tube. The cell pellet is resuspended in 2 ml of medium. Then the cells are counted for viability and total count using Trypan Blue Stain (Cat. No. 15250-161, Gibco Life Technologies) in a hemocytometer counting chamber. HUVEC are resuspended at a concentration of 1.4×10⁵ cells/ml in EGM medium. The fibronectin is removed from collagen coated plates immediately before adding the HUVEC suspension. A 100 μl of the HUVEC suspension is added to each appropriate collagen coated well, and then the plate is placed at 37° C. in a CO₂ incubator. The plates are incubated for 3 days changing medium 2× during these 3 days. ¹ Preparation of HUVEC: 1 vial of HUVEC cells is thawed at room temperature. EGM medium (25 ml) is placed in T75 flask. HUVEC is added to medium in flask and is grown to confluency (approximately 5 days). ² 8 ml Vitrogen, 1 ml Medium 199 (10×), and 5 ml 0.1 N NaOH are gently mixed in 50 ml tube so as not to cause air bubbles in mixture. ³ 1 ml of warm PBS (Cat. No. 14190-136 Gibco Life Technologies) is added to vial of fibronectin (human, 1 mg) and mixed thoroughly. 100 μl stock+1900 μl PBS=50 μg/ml working stock.

Neutrophil Purification

On the day of the transendothelial migration assay (TEM) neutrophils are purified from human whole blood. The blood is obtained from a cubital vein by conventional venipucture performed by a qualified nurse. The blood is collected in four 10 ml EDTA vacutainer tubes (Cat. No. 366457, VWR). Once the whole blood is collected the neutrophils are purified by using an established double gradient density (Histopaque, Cat. No. 1077-1 and Cat. No. 1119-1, Sigma) centrifugation method. Once the neutrophils are collected they are resuspended in cold phosphate buffered saline (PBS) which contains 0.2% glucose (Cat. No. G7021, Sigma) and 0.1% human serum albumin (HSA) (albumin, human, 25%, USP, Genesis Bio-Pharmaceuticals, Inc.) and repelleted by centrifuging at 1800 rpm for 10 minutes. Neutrophils are lysed to remove any red blood cell (RBC) contamination by gently adding 6 ml of cold Milli-Q filtered H₂O for 30 seconds and 3 ml of cold filtered 2.7% NaCl (Cat. No. S5886, Sigma) to the pellet. This step may need to be conducted two or three times. The cell count and viability of the neutrophils are determined using Trypan Blue Stain in a hemocytometer counting chamber.

Transendothelial Migration Assay (TEM)

During the purification period of the neutrophils, the HUVEC monolayer is induced for adhesion molecule expression with 300 U/ml of Tumor Necrosis Factor-α(TNF, Cat. No. 1371-843, Boehringer Mannheim) for approximately 4 hours. Once the neutrophils are purified they are then labeled using carboxyfluorescein (CFSE, Cat. No. C-1157, Molecular Probes) in dimethyl sulphoxide (DMSO, Cat. No. D2650, Sigma) for 20 minutes on ice⁴. The neutrophils are then washed (3×) with cold PBS which contains glucose and HSA to remove any excess dye. Following washing, the neutrophils are resuspended in warm complete EGM at a concentration of 1×10⁶ cells/ml. The neutrophils are now incubated for approximately 30 minutes with 300 U/ml of TNF-α at 4° C. During the incubation time the compounds or the control blocking antibodies (CD11b antibody—Cat. No. 347550, Becton Dickinson; LFA-1/Beta Chain CD18 antibody—Cat. No. M0783, DAKO) which are to be tested are prepared. The concentrations to be tested of each appropriate compound is prepared in EGM medium. Once the compound or antibody concentrations are prepared an aliquot of each compound concentration and/or antibody is placed in a well of a deep well Dynablock 1000 polystyrene plate (1.0 ml, non-sterile, Cat. No. 40002-006 and caps, plate cover, non-sterile, Cat. No. 40002-000, VWR). Following the incubation period of the neutrophils with the TNF-α the neutrophils are added in aliquots to the compounds and/or antibodies in the deep well plate. The neutrophils now with compound and/or antibodies in the deep well plate are incubated at 4° C. for 30 minutes. While the neutrophils and compounds are incubating the 96 well plates which contain the HUVEC monolayer and TNF-α are washed (3×) with warm EBM medium to remove the TNF-α. Now the neutrophils with compound and/or anbibodies in 100 μl aliquots are added to the HUVEC monolayer in the 96 well tissue culture plate. Once the neutrophils with compound and/or antibodies are added to the HUVEC the plate is then incubated for 30 minutes at 37° C. in a CO₂ incubator. This mode is used for evaluating migration. After incubation the plates are scanned (Scan 1) on a PerSeptive Biosystems, CytoFlour 4000; excitation filter 485/20, emission filter 530/25, at a gain of 50. Once scanned the non-adherent or non-migrated neutrophils are removed by using an automated Titertek Plus MRD8 plate washer (ICN Pharmaceuticals, Inc.). Warm Hanks Balanced Salt Solution (HBSS, Cat. No. 14175-095, Gibco Life Technologies)⁵ with 1 mM (ethylene glycol-bis[b-aminoethyl ether]N,N,N′N′-tetraacetic acid) (EGTA, Cat. No. E3889, Sigma) is used for washing which dislodges the adhered neutrophils by chelating the divalent cations which is absolutely essential for binding of neutrophils to ICAM-1 expressing endothelial cells. Following washing the plate is scanned again at the same settings as above (Scan 2). ⁴ Once neutrophils are purified and ready for labeling, the pellet is resuspended in 5 ml cold PBS buffer containing 0.2% glucose and 0.1% HSA. 16 μl of 10 mM CFSE solution is added to the 5 ml cell suspension. The tube is covered with aluminum foil. The tube is placed on ice or at 4° C. for 20 minutes. (10 mM stock CFSE in sterile DMSO=5.5747 mg CFSE in 1 ml DMSO.) ⁵ 1 mM EGTA in HBSS=190.2 mg EGTA in 500 ml

Calculation of Data

The data is processed in a prepared EXCEL spreadsheet where the background of the plate is subtracted from all wells and the % TEM is calculated by dividing the counts from Scan 2 (number of migrated cells) by the counts from Scan 1 (total number of cells) and multiplying the result by 100.

To express the activity of a compound the % Reduction is calculated using the following formula:

$$\% Reduction = \frac{( 1 - ( \% Total Compound ) - ( \% Total No Ab / No TNF ) )}{( \% Total No Ab / TNF ) - ( \% Total No Ab / No TNF )}$$

This data is processed in a prepared EXCEL spreadsheet with each component of the formula as follows:

% Reduction=Activity of Compound

% Total Compound=% Total Cells Migrated from cells treated with test compound

% Total No Ab/No TNF=% Total Cells Migrated from cells that were treated with no antibody or compound and were not induced with TNF

% Total No Ab/TNF=% Total Cells Migrated from cells that were treated with no antibody or compound and were induced with TNF)

Statistics are performed using the two-sided t-test with equal variance in EXCEL and the results are recorded as the P value.

To find the concentration of a compound that inhibits 20% or 50% of the neutrophils from migrating to upregulated HUVEC, an Inhibitory Concentration (IC₂₀ or IC₅₀) is calculated. Sigma Plot is used to calculate the IC by plotting concentration (x axis) vs % Reduction (y axis) and fitting a line to the data.

In addition to the TEM assay described above, additional assays include a rat reperfusion model as follows.

Rat Myocardial Infarct/Reperfusion Injury Model

Surgical Preparation of Rats:

Male, Sprague-Dawley rats are anesthetized with urethane, 1.25 g/kg ip. A carotid artery and jugular vein are exteriorized and cannulated with PE-50 tubing for recording blood pressure and to facilitate intravenous administration of dye or drug. A Tracheotomy is performed. The animals are connected to a ventilator and ventilated with oxygen to maintain physiological blood pH, PO2, and PCO2. Needle electrodes are placed for a lead II electrocardiogram. The animals are maintained at 37° C. by means of electric heating pads adjusted to the desired temperature and controlled via a rectal thermistor probe and controller. The heart is carefully isolated by a left thoracotomy at the fifth intercostal space, and the left anterior descending coronary artery (LAD) is located. A ligature of 6-0 silk is placed around the LAD, with the ends threaded through a small length of PE-320 tubing to facilitate rapid occlusion and reperfusion of the artery. The LAD is occluded by clamping the suture and tubing tight against the heart surface using 25 mm Schwarz aneurysm clip. Occlusion lasts for 90 min and is followed by reperfusion for 3.0-4.5 hr. Animals are dosed with drug or vehicle 10 min prior to reperfusion of the affected area of the heart by intravenous delivery via a jugular vein. Sham-operated rats are not subjected to ischemia or reperfusion. At the end of the experiment, the LAD is permanently re-occluded and a 10 mg/ml solution of Evans Blue Stain is administered via the jugular cannula to identify the area affected by ischemia, i.e., the area-at-risk (AAR). The stained heart is rapidly excised and placed into 0.9% saline at 4° C. prior to the determination of creatine phosphokinase activity (CPK).

Determination of Creatine Phosphokinase Activity:

The left ventricular free wall (LVFW) is dissected free from the heart and weighed. The AAR, as defined by the absence of stain, is dissected from the LVFW and also weighed. The AAR is homogenized for 5 sec in 4 ml of 0.25 M sucrose containing 1 mM EDTA and 10 mM mercaptoethanol at 4° C. The homogenate is centrifuged at 30,000×g for 30 min at 4° C. The supernatant is decanted for determination of CPK activity and the pellet is stored frozen for the isolation and assay of myeloperoxidase activity. CPK activity is assayed spectrophotometrically with a commercially supplied substrate, CPK Assay Vial® (Sigma Diagnostics), at a wavelength of 340 nm at 24-26° C.

Determination of Myeloperoxidase Activity:

Myeloperoxidase (MPO) is isolated from the frozen pellet after the preparation of CPK. The pellet is suspended in 50 mM phosphate buffer, pH 6, containing 0.5% hexadecyltrimethylammonium bromide (HTAB) to a concentration of approximately 10% sonicated for 10 sec and frozen on dry ice. Three freeze-thaw cycles are done with 10 sec of sonication between cycles. The samples are chilled on ice for 30 min followed by centrifugation at 12,500×g for 15 min at 4° C. An aliquot of the supernatant is assayed spectrophotometrically for MPO activity in 50 mM sodium phosphate buffer, pH 6, containing 0.167 mg/ml o-dianisidine dihydrochloride and 0.0005% hydrogen peroxide at a wavelength of 460 nm at 24-26° C.

Calculations and Statistical Analysis:

The results are reported as the mean ±SEM. CPK and MPO activity are expressed as units/g tissue, where 1 unit of CPK activity is defined as the quantity of CPK utilizing 1 μmol peroxide per minute. The AAR is quantified as a percentage of the LVFW based on weight. Mean arterial blood pressure (MABP) is calculated as one-third the difference between systolic and diastolic blood pressure added to diastolic blood pressure. Data are analyzed for statistical significance of treatment effects at the 95% confidence level by a pooled t-test or by one-way analysis of variance.

Composition and Method Examples

The following non-limiting examples illustrate the subject invention. The following composition and method examples do not limit the invention, but provide guidance to the skilled artisan to prepare and use the compounds, compositions, and methods of the invention. In each case other compounds within the invention may be substituted for the example compound shown below with similar results.

### Example A

Pharmaceutical compositions in the form of an intravenous solution are prepared by conventional methods, such as mixing the following:

| | |
|---|---|
| Ingredient | Quantity (mls) |

| | | |
|---|---|---|
| Compound of Example I | 1 | mg. |
| Sterile water | 10 | ml |

| | |
|---|---|
| HCL and/or NaOH | pH 7.2-7.5 |

When 1 ml of the above composition is administered intravenously, either immediately before or immediately after a tissue damage event (aneurysm repair, coronary bypass, transplant surgery, traumatic hemorrhage, organ ischemia due to hypoperfusion, sepsis, etc.), tissue damage is avoided or reduced.

### Example B

Pharmaceutical compositions in liquid form are prepared by conventional methods, formulated as follows:

| | |
|---|---|
| Ingredient | Quantity |

| | | |
|---|---|---|
| Compound of Example II or III | 1 | mg |
| Phosphate buffered physiological saline | 10 | ml |
| Methyl Paraben | 0.05 | ml |

When 1.0 ml of the above composition is administered subcutaneously, either immediately before or immediately after a tissue damage event (aneurysm repair, coronary bypass, transplant surgery, traumatic hemorrhage, organ ischemia due to hypoperfusion, sepsis, etc.), tissue damage is avoided or reduced.

While particular embodiments of the subject invention have been described, it would be obvious to those skilled in the art that various changes and modifications to the compositions disclosed herein can be made without departing from the spirit and scope of the invention. It is intended to cover, in the appended claims, all such modifications that are within the scope of this invention.

What is claimed is:

1. A compound having the structure: 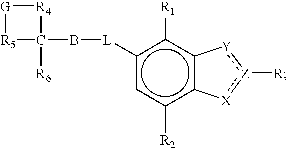
wherein X and Y are heteroatoms wherein at least X or Y is nitrogen wherein the nitrogen can be unsubstituted or substituted with a lower alkyl group;
Z is selected from the group consisting of a carbon atom, two carbon atoms or a heteroatom;
R is independently selected from the group consisting of alkyl, lower alkyl, aromatic ring, carbocyclic aliphatic ring, halo, haloalkyl, heteroalkyl, lower heteroalkyl, heteroaromatic ring, heterocyclic aliphatic ring, hydrogen, —OH, —NH₂, —SH, —OCH₃;
R₁ and R₂ are independently, selected from the group consisting of alkyl, lower alkyl, aromatic ring, carbocyclic aliphatic ring, halo, haloalkyl, heteroalkyl, lower heteroalkyl, heteroaromatic ring, heterocyclic aliphatic ring, or hydrogen;
L is selected from the group consisting of 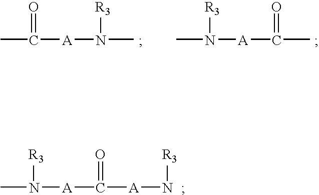
wherein A is selected from the group consisting of a branched or unbranched alkyl, a branched or unbranched lower alkyl, or A is a covalent bond;
R₃ is selected from the group consisting of alkyl, lower alkyl, aromatic ring, carbocyclic aliphatic ring, haloalkyl, heteroalkyl, lower heteroalkyl, heterocyclic aliphatic ring, or hydrogen;
B is selected from the group consisting of alkyl, lower alkyl, haloalkyl, heteroalkyl, lower heteroalkyl or B is a covalent bond;
G is nil, or a substituent that links R₄ and R₅ into a cyclic ring structure which may be a 5-10 atom aromatic, aliphatic, heteroaromatic, or heteroaliphatic ring structure, which is unsubtituted or substituted wherein R₄ is a substituent at any position on the ring structure;
if G is nil then, R₄ and R₅ are:
R₄ is: 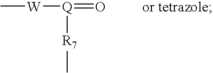
wherein Q is selected from the group consisting of a carbon atom, 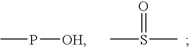
W is selected from the group consisting of —OH or —NHOH;
R₇ is selected from the group consisting of alkyl, lower alkyl, aromatic ring, heteroaromatic ring, carbocyclic aliphatic ring, heteroalkyl, lower heteroalkyl, heterocyclic aliphatic ring, hydrogen ora covalent bond;
R₅ is selected from the group consisting hydrogen, alkyl, lower alkyl, aromatic ring, carbocyclic aliphatic ring, halo, haloalkyl, heteroalkyl, lower heteroalkyl, heterocyclic aliphatic ring, —C₂H₄— aromatic ring, —C₂H₄-carbocyclic aliphatic ring, —C₂H₄-heterocyclic aliphatic ring, —C₂H₄—Ph, —CH₂-aromatic ring, —CH₂-carbocyclic aliphatic ring, —CH₂-heterocyclic aliphatic ring, —CH₂Ph;
R₆ is selected from the group consisting of alkyl, lower alkyl, aromatic ring, carbocyclic aliphatic ring, halo, haloalkyl, heteroalkyl, lower heteroalkyl, heterocyclic aliphatic ring, hydrogen,, or any R₄ group;
optical isomers, diastereomers, and enantiomers thereof, and mixtures thereof, and pharmaceutically-acceptable salts, hydrates, biohydrolyzable amides, esters, and imides thereof.

2. The compound of claim 1 wherein both X and Y are nitrogen.

3. The compound of claim 2 wherein B is lower alkyl, lower heteroalkyl, or a covalent bond.

4. The compound of claim 3 wherein R is selected from the group consisting of alkyl, lower alkyl, aromatic ring, carbocyclic aliphatic ring, halo, haloalkyl, heteroalkyl, lower heteroalkyl, heterocyclic aliphatic ring, heteroaromatic ring, hydrogen, —OH, —NH₂, or —OCH₃.

5. The compound of claim 4 wherein R₁ and R₂ are, independently, selected from the group consisting of lower alkyl, hydrogen, heteroalkyl, a 5 or 6-membered heterocyclic aliphatic ring, heteroalkyl, a branched or nonbranched alkyl heteroaromatic ring, or phenyl group.

6. The compound of claim 5 wherein R₁ and R₂ are, independently, selected from the group consisting of lower alkyl, hydrogen, heteroalkyl, a branched alkyl heteroaromatic ring wherein the alkyl chain contains a heteroatom, or a phenyl group.

7. The compound of claim 6 wherein A is an unbranched C₁ to C₄ alkyl and R₃ is alkyl or hydrogen.

8. The compound of claim 4 wherein G is nil.

9. The compound of claim 8 wherein Q is a carbon atom and W is an —OH group.

10. The compound of claim 9 wherein R₇ is selected from the group consisting of phenyl, C₁-C₄ alkyl, hydrogen, or a covalent bond.

11. The compound of claim 10 wherein R₅ is selected from the group consisting carbon atom, —C₂H₄— aromatic ring, —C₂H₄-carbocyclic aliphatic ring, —C₂H₄-heterocyclic aliphatic ring, —C₂H₄—Ph, —CH₂-aromatic ring, —CH₂-carbocyclic aliphatic ring, —CH₂-heterocyclic aliphatic ring, —CH₂Ph; or a phenyl group.

12. The compound of claim 1 wherein if R is —SH then R₅ is selected from the group consisting of C₁-C₁₇ alkyl, hydrogen, lower alkyl, aromatic ring, carbocyclic aliphatic ring, halo, haloalkyl, heteroalkyl, lower heteroalkyl, heterocyclic aliphatic ring, —C₂H₄— aromatic ring, —C₂H₄-carbocyclic aliphatic ring, —C₂H₄-heterocyclic aliphatic ring, —C₂H₄—Ph, —CH₂-aromatic ring, —CH₂-carbocyclic aliphatic ring, —CH₂-heterocyclic aliphatic ring, —CH₂Ph and R₆ is selected from the group consisting of C₁-C₁₇ alkyl, lower alkyl, aromatic ring, carbocyclic aliphatic ring, halo, haloalkyl, heteroalkyl, lower heteroalkyl, heterocyclic aliphatic ring, hydrogen,, or any R₄ group.

13. A compound having the structure: 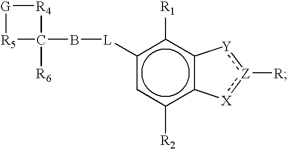
wherein X and Y are both nitrogen wherein the nitrogen can be unsubstituted or substituted with a lower alkyl group;
Z is a single carbon atom;
R is selected from the group consisting of alkyl, lower alkyl, aromatic ring, carbocyclic aliphatic ring, halo, haloalkyl, heteroalkyl, lower heteroalkyl, heteroaromatic ring, heterocyclic aliphatic ring, hydrogen, —OH, —NH₂, —SH, or —OCH₃;
R₁ and R₂ are, independently, selected from the group consisting of alkyl, lower alkyl, aromatic ring, carbocyclic aliphatic ring, halo, haloalkyl, heteroalkyl, lower heteroalkyl, heteroaromatic ring, heterocyclic aliphatic ring, or hydrogen; 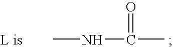
B is selected from the group consisting of alkyl, lower alkyl, haloalkyl, heteroalkyl, lower heteroalkyl or B is a covalent bond;
G is nil; 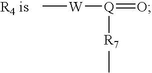
wherein Q is selected from the group consisting of a carbon atom, 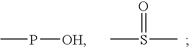
W is selected from the group consisting of —OH or —NHOH;
R₇ is selected from the group consisting of alkyl, lower alkyl, aromatic ring, heteroaromatic ring, carbocyclic aliphatic ring, heteroalkyl, lower heteroalkyl, heterocyclic aliphatic ring, hydrogen oror a covalent bond;
R₅ is selected from the group consisting of hydrogen, alkyl, lower alkyl, aromatic ring, carbocyclic aliphatic ring, halo, haloalkyl, heteroalkyl, lower heteroalkyl, heterocyclic aliphatic ring, —C₂H₄— aromatic ring, —C₂H₄-carbocyclic aliphatic ring, —C₂H₄-heterocyclic aliphatic ring, —C₂H₄—Ph, —CH₂-aromatic ring, —CH₂-carbocyclic aliphatic ring, —CH₂-heterocyclic aliphatic ring, —CH₂Ph;
R₆ is selected from the group consisting of alkyl, lower alkyl, aromatic ring, carbocyclic aliphatic ring, halo, haloalkyl, heteroalkyl, lower heteroalkyl, heterocyclic aliphatic ring, hydrogen,, or any R₄ group;
optical isomers, diastereomers, and enantiomers thereof, and mixtures thereof, and pharmaceutically-acceptable salts, hydrates, biohydrolyzable amides, esters, and imides thereof.

14. The compound of claim 13 wherein B is a C₁-C₄ alkyl, C₁-C₄ heteroalkyl interrupted with an oxygen atom, or a covalent bond.

15. The compound of claim 14 R is selected from the group consisting of alkyl, lower alkyl, aromatic ring, carbocyclic aliphatic ring, halo, haloalkyl, heteroalkyl, lower heteroalkyl, heterocyclic aliphatic ring, heteroaromatic ring, hydrogen, —OH, —NH₂, or —OCH₃.

16. The compound of claim 15 wherein R is selected from the group consisting of alkyl, lower alkyl, aromatic ring, carbocyclic aliphatic ring, halo, haloalkyl, heteroalkyl, lower heteroalkyl, hydrogen, —OH, —NH₂, —OCH₃.

17. The compound of claim 15 wherein R₁ and R₂ are, independently, selected from the group consisting of lower alkyl, hydrogen, a 5 or 6-membered heterocyclic aliphatic ring, a branched or nonbranched alkyl heteroaromatic ring, or a phenyl group.

18. The compound of claim 17 wherein R₁ and R₂ are, independently, selected from the group consisting of lower alkyl, hydrogen, a branched alkyl heteroaromatic ring wherein the alkyl chain contains a heteroatom, or a phenyl group.

19. The compound of claim 17 wherein Q is a carbon atom and W is an —OH group.

20. The compound of claim 17 wherein R₇ is selected from the group consisting of phenyl, C₁-C₄ alkyl, hydrogen, or a covalent bond.

21. The compound of claim 18 wherein R₇ is selected from the group consisting of methyl, hydrogen or a covalent bond.

22. The compound of claim 20 wherein R₅ is selected from the group consisting carbon atom, —C₂H₄— aromatic ring, —C₂H₄-carbocyclic aliphatic ring, —C₂H₄-heterocyclic aliphatic ring, —C₂H₄—Ph, —CH₂-aromatic ring, —CH₂-carbocyclic aliphatic ring, —CH₂-heterocyclic aliphatic ring, —CH₂Ph; or a phenyl group.

22. The compound of claim 13 wherein if R is —SH then R₅ is selected from the group consisting of C₁-C₁₇ alkyl, hydrogen, lower alkyl, aromatic ring, carbocyclic aliphatic ring, halo, haloalkyl, heteroalkyl, lower heteroalkyl, heterocyclic aliphatic ring, —C₂H₄— aromatic ring, —C₂H₄-carbocyclic aliphatic ring, —C₂H₄-heterocyclic aliphatic ring, —C₂H₄—Ph, —CH₂-aromatic ring, —CH₂-carbocyclic aliphatic ring, —CH₂-heterocyclic aliphatic ring, —CH₂Ph and R₆ is selected from the group consisting of C₁-C₁₇ alkyl, lower alkyl, aromatic ring, carbocyclic aliphatic ring, halo, haloalkyl, heteroalkyl, lower heteroalkyl, heterocyclic aliphatic ring, hydrogen, or any R₄ group.

23. A pharmaceutical composition comprising:
(a) a safe and effective amount of a compound of claim 1 or 13; and
(b) a pharmaceutically-acceptable excipient.

24. A method of preventing or treating undesirable or abnormal inflammatory response comprising administering to a human or lower animal in need thereof, a safe and effective amount of a compound of claim 1 or 13.

25. The method of claim 24 wherein the inflammatory response is chronic inflammatory lung disease.

26. The method of claim 24 wherein the inflammatory response is ischemia-reperfusion injury.

27. The method of claim 24 wherein the inflammatory response is arthritis.

28. The method of claim 24 wherein the inflammatory response is inflammatory bowel disease.

29. The method of claim 24 wherein the inflammatory response is hepatitis, pancreatitis, allergy, gout, radiation induced, ulcer, fibrosis, or migraine.

30. The method of claim 24 wherein the inflammatory response is inflammatory skin disease.

31. The method of claim 24 wherein the inflammatory response cardiovascular disease or vascular disease.
